SEQUENCE LISTING (2 sequences)

SEQ ID NO: 1 - DNA, 1017 bp, synthetic construct
  source 1..1017: mol_type = other DNA
atgacaaacactttcaccaccctgctggagaccaaagacgccctgcttgcggatggggccaccggcaccaacctgttcaacatgggcctccagtccggtgatgcgccggagctgtggaatgtggatgaacccaagaaaatcaccgcgctctatcagggggcggtcgatgcgggcagcgatctgttcctgaccaatacctttggcgggaccgccgcgcggctgaagctgcacgacgcccaccgccgggtccgggagctgaacgtcgcgggggccgagttgggccgcaacgtcgcggatcgctctgagcgcaagatcgccgtggccggatcagtcggaccgactggcgaaatcatgcagccggtgggtgaactgagccacgcgctcgccgtggaaatgttccatgagcaggccgaggcgctgaaagagggcggcgtcgacgtgttgtggctggagacgatctctgctccggaagagtaccgcgccgccgctgaagcgttcaaactggcggatatgccctggtgcggcaccatgagttttgacaccgccgggcgcaccatgatgggggtcacctccgccgatatggcgcagctggtcgaggagttcgacccagcgcctctggcctttggtgccaattgcggcaccggggcgtccgacattctgcgcacggtacttgggttcgccgcccagggcacgacccgcccgatcatttccaagggcaatgccgggatcccgaaatatgtcgatggtcatatccactatgacggcacgccgaccctgatgggggaatatgcagccatggcaagagattgcggcgccaaaatcattggtggctgctgtggcaccatgccggatcacctgcgcgccatgcgcgaggcgctggatacccgcccccggggcgagcagctaacactggagcggatcgttgaggtgcttggtcccttcacctccgacagtgacggcaccggtgaggatacagcccctgaccgccgcagccgtcgcggtcgccgtcgcggctga

SEQ ID NO: 2 - protein, 338 aa, synthetic construct
MTNTFTTLLETKDALLADGATGTNLFNMGLQSGDAPELWNVDEPKKITALYQGAVDAGSDLFLTNTFGGTAARLKLHDAHRRVRELNVAGAELGRNVADRSERKIAVAGSVGPTGEIMQPVGELSHALAVEMFHEQAEALKEGGVDVLWLETISAPEEYRAAAEAFKLADMPWCGTMSFDTAGRTMMGVTSADMAQLVEEFDPAPLAFGANCGTGASDILRTVLGFAAQGTTRPIISKGNAGIPKYVDGHIHYDGTPTLMGEYAAMARDCGAKIIGGCCGTMPDHLRAMREALDTRPRGEQLTLERIVEVLGPFTSDSDGTGEDTAPDRRSRRGRRRG

(19) United States
(12) Patent Application Publication
(10) Pub. No.: US 2026/0015574 A1
(43) Pub. Date: Jan. 15, 2026

(54) METHODS FOR SHORTENING LAG PHASE DURATION IN MICROORGANISMS

(71) Applicant: YEDA RESEARCH AND DEVELOPMENT CO. LTD., Rehovot (IL)

(72) Inventors: Einat SEGEV, Rehovot (IL); Martin SPERFELD, Rehovot (IL); Delia NARVAEZ, Rehovot (IL)

(21) Appl. No.: 18/837,677

(22) PCT Filed: Feb. 16, 2023
(86) PCT No.: PCT/IL2023/050169

§ 371 Date: Aug. 12, 2024

Related U.S. Application Data
(60) Provisional application No. 63/310,600, filed on Feb. 16, 2022.
(60) Provisional application No. 63/356,575, filed on Jun. 29, 2022.

(51) Int. Cl. C12N 1/38; C12N 9/10; C12N 15/74

(52) U.S. Cl. CPC C12N 1/38; C12N 9/1007; C12N 15/74; C12Y 201/01005

(57) ABSTRACT

The present invention is directed to a method for shortening lag phase of a microorganism, including contacting the microorganism with an effective amount of a methyl group donor and/or one-carbon group donor, thereby shortening the lag phase of the microorganism.

### CROSS-REFERENCE TO RELATED APPLICATIONS

This application claims the benefit of priority of U.S. Provisional Patent Applications Nos. 63/310,600, titled “METHYLATED COMPOUNDS DETERMINE THE LAG PHASE DURATION IN (MARINE) BACTERIA”, filed Feb. 16, 2022, and 63/356,575, titled “METHODS FOR SHORTENING LAG PHASE DURATION IN MICROORGANISMS”, filed Jun. 29, 2022, the contents of which are all incorporated herein by reference in their entirety.

### REFERENCE TO AN ELECTRONIC SEQUENCE LISTING

The contents of the electronic sequence listing (YEDA-P-016-PCT.xml; Size: 4,141 bytes; and Date of Creation: Feb. 15, 2023) is herein incorporated by reference in its entirety.

### FIELD OF INVENTION

The present invention is in the field of molecular and cellular biology, and specifically is directed to, inter alia, methods of shortening lag phase of microorganisms.

### BACKGROUND OF THE INVENTION

Marine heterotrophic bacteria account for circa 20% of the global ocean biomass. The bacteria are “master recyclers” of the ocean and consume organic carbon that is mainly produced by photosynthetic organisms. However, photosynthetic carbon production is not constant, and photosynthetic organisms, which are here referred to as microalgae, are subjected to diurnal and seasonal fluctuations. Consequently, bacteria must endure prolonged phases of starvation under conditions of low microalgal productivity, as they occur for example at night or in winter. On the other hand, the bacteria require to rapidly activate their metabolism with the onset of microalgal productivity. An early response is vital for the bacteria to outgrow co-occurring heterotrophs that compete for the same resources.

The time bacteria require to respond to available resources is manifested by the duration of the lag phase. The lag phase was recently revisited and as pointed out, it is a dynamic, organized, adaptive and evolvable process. During the lag phase, bacteria synthesize bottleneck proteins that are required for utilizing available metabolites. Those bottleneck proteins are involved in the production of energy, and the formation of limiting building blocks. In accordance, the external addition of limiting building blocks alleviates metabolic needs during the lag phase. Such limiting building blocks are a valuable currency that is exchanged between interacting microorganisms. The lag phase was largely studied in human pathogens and in food-spoiling bacteria. However, much is unknown about the bacterial lag phase in an environmental context.

Several groups of marine heterotrophic bacteria, such as the Roseobacter group, appear to be adapted to efficiently consume compounds that are secreted by microalgae. Roseobacters are ubiquitous across marine surface waters and are commonly found in association with abundant microalgae.

There is still a great need for compounds and methods for shortening lag phase of microorganisms.

### SUMMARY OF THE INVENTION

The present invention, in some embodiments, is based, at least in part, on the findings that minute amounts of methylated compounds produced by microalgae, as well as one-carbon group donor(s) induce a marked shortening of the lag phase in Phaeobacter inhibens.

Further, the present invention, in some embodiments, is based, at least in part, on the findings that methylated compounds also shorten the lag phase in various marine bacteria, as well as in non-marine model bacteria including strains of Bacillus, Vibrio, and Escherichia. Thus, the current findings unveil a fundamental mechanism in bacterial lag phase regulation and open new opportunities to control the growth of harmful bacteria and/or encourage growth of beneficial bacterial in a variety of applications.

According to one aspect, there is provided a method for shortening lag phase of a microorganism, the method comprising contacting the microorganism with an effective amount of a methyl group donor, thereby shortening lag phase of the microorganism.

In some embodiments, the methyl group donor comprises a tertiary sulfonium group or a quaternary ammonium group.

In some embodiments, the methyl group donor comprises one or more methyl groups.

In some embodiments, the one or more methyl groups is covalently bound to a sulfate/sulfur atom or a nitrogen atom.

In some embodiments, the methyl group donor is selected from the group consisting of: dimethylsulfoniopropionate (DMSP), betaine, choline, dimethylsulfonioacetate (DMSA), carnitine, homarine, stachydrine, trigonelline, gonyol, S-methylmethionine (SMM), and any combination thereof.

In some embodiments, the methyl group donor comprising a tertiary sulfonium group is selected from the group consisting of: DMSP, DMSA, gonyol, and any combination thereof.

In some embodiments, the methyl group donor comprising a quaternary ammonium group is selected from the group consisting of: betaine, choline, homarine, carnitine, stachydrine, trigonelline, and any combination thereof.

In some embodiments, the method further comprises a step comprising subjecting the microorganism to suboptimal salt concentration, suboptimal temperature, or both.

In some embodiments, the microorganism is a transgenic cell or a transformed cell.

In some embodiments, the transgenic cell or transformed cell heterologously expresses a polynucleotide encoding a betaine-homocysteine S-methyltransferase (Bmt).

In some embodiments, the microorganism is selected from the group consisting of: bacterium, fungus, microalga, and any combination thereof.

In some embodiments, the fungus is a yeast.

In some embodiments, the method further comprises a step preceding the contacting, comprising transfecting or transforming the microorganism with a polynucleotide encoding a Bit.

In some embodiments, the polynucleotide comprises a nucleic acid sequence set forth in SEQ ID NO: 1, or a functional analog thereof having at least 80% sequence homology thereto.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1D include graphs, a non-limiting scheme, and chemical structures showing the identification of metabolic reactions that are elicited in the bacterium P. inhibens during co-cultivation with the algal host Emiliania huxleyi. Dual RNA-sequencing revealed that bacterial genes involved in methyl group metabolism were upregulated (1A; blue number) and among the highest expressed metabolic genes in the presence of algae (1B; blue bars). The upregulated genes encode for enzymes involved in harvesting, dissimilating and assimilating methyl groups from donor molecules (1C). Numbers given in blue, red and grey indicate upregulated, non-regulated, and downregulated genes, respectively, and match throughout the document. Chemical structures of discussed molecules are depicted in 1D.

FIGS. 2A-2B include RNA sampling points of bacterial pure cultures (P. inhibens) and bacteria in co-culture with algae (P. inhibens+E. huxleyi). The RNA sampling points encompass the exponential and stationary phase of bacteria grown in pure culture with glucose (2A; light brown, glucose 1 and 2, respectively), as well as the different interaction phases of bacteria in co-culture with algae (2A; dark brown, co-cultures day 04-day 11/12). RNA samples were subjected to dual RNA-Sequencing and differential gene expression analysis (results shown in FIGS. 1A-1C, and 3-4). The growth of algae is stimulated by bacteria during the early interaction phase, and harmed by bacteria during the late interaction phase (2B; light green: algae without bacteria; dark green: algae with bacteria).

FIG. 3 includes a plot showing the characterization of bacterial gene transcription profiles in the presence and absence of algae. Bacterial transcription profiles were similar in the presence of living algae (co-cultures day 04-09), but shifted when algae commenced death (co-culture day 11/12), or were entirely absent (glucose 1 and 2). Samples of co-cultures from day 04-09 were treated as replicates for differential gene expression analysis, and were compared to samples of bacteria grown exponentially with glucose (glucose 1). This allowed the identification of bacterial genes that are robustly regulated.

FIG. 4 includes a heatmap showing transcript abundances of bacterial methyl group metabolism genes. The abundances were similar in the presence of living algae (co-cultures day 04-09), but shifted when algae commenced death (co-culture day 11/12), or were entirely absent (glucose 1 and 2). Rows were sorted by highest mean transcript abundance in co-cultures of day 04-09 (log 2 TPM; transcripts per million). Row names include the gene number given in FIG. 1C, the RefSeq gene locus tag accession number, and the RefSeq product description.

FIGS. 5A-5C include graphs showing the effect of the methylated compound DMSP on the growth of the bacterium P. inhibens. Growth experiments revealed that small amounts of DMSP stimulate the bacterium (5A) and shorten its lag time (5B). It was further determined that DMSP shortens the lag time in the nanomolar concentration range (5C). Growth experiments were conducted in artificial seawater medium with 1 mM glucose as substrate.

FIG. 6 includes a graph showing temporal changes within two distinct P. inhibens populations that occur during the lag phase. Imaging flow cytometry revealed that bacteria mainly occur as aggregates (orange bars) at the beginning of the lag phase. During the lag phase, the abundance of single-celled bacteria increases. The transition from aggregates to single-celled bacteria is concluded after 3 h in DMSP-treated cultures (blue bars), but takes 4 h in control cultures (grey bars). This confirms that DMSP influences the lag phase of bacteria.

FIGS. 7A-7C include graphs and chemical structures showing the identification of the chemical property that induces the lag phase shortening effect. Growth experiments revealed that the methyl group moieties of DMSP are required for lag phase shortening, and that the extent of the effect correlates with the amount of methyl groups attached per molecule (7A). Growth experiments further revealed that a variety of methylated compounds shorten the lag phase (7B). By generating P. inhibens knock-out mutant strains, it was found that the dmdA gene, which encodes for a DMSP demethylase enzyme (FIG. 1C, gene 25), is required for lag phase shortening with DMSP, while the mttB1 gene (FIG. 1C, gene 3), which encodes for a betaine demethylase enzyme, is required for lag phase shortening with betaine (7C).

FIG. 8 includes growth curves of the bacterium P. inhibens grown in the presence of: betaine, choline, dimethylglycine (DMG), sarcosine, glycine, alanine, acetate, stachydrine, proline, trigonelline, homarine, nicotinate, carnitine, gonyol, DMSA, cysteine, methanethiol, methanol, serine, or methionine. The growth curves were used to calculate Δlag times shown in FIG. 7A-7B, and doubling times shown in FIG. 9. Cultivations were done as quadruplicates. Control: bacteria grown with 1 mM glucose (grey points); Treatment: bacteria grown with 1 mM glucose and 2 μM of the respective compound (blue points).

FIG. 9 includes a graph showing doubling times of the bacterium P. inhibens grown in the presence of the indicated compounds.

FIGS. 10A-10B include a non-limiting scheme and graphs showing the metabolic response of the bacterium P. inhibens towards DMSP during the lag phase. DMSP induced the transcription of methyl group metabolism genes that are involved in harvesting methyl groups from DMSP (10A, gene 4, 7, 8, 9, 17, 25), in dissimilating methyl groups for ATP generation (10A, gene 29), and in assimilating methyl groups via the methionine cycle (10A, gene 83). Numbers given in blue, red and grey indicate genes that are upregulated, non-regulated, or downregulated in the presence of DMSP compared to the control condition, respectively (within the first 15 min of the lag phase). When administering DMSP to stationary phase bacteria, the cells responded with increased methionine synthesis activity (10B). This activity was measured by incubating protein crude extracts of stationary phase bacteria with DMSP and homocysteine, and measuring the enzymatic products methionine and 3-MMPA using LC/MS.

FIGS. 11A-11B include graphs showing methyl group metabolism genes that are differentially expressed in DMSP treated lag phase bacteria, compared to untreated control conditions. The transcriptional response was measured within the first 15 min (11A) and 40 min (11B) of the lag phase. Numbers given in blue and red and indicate genes that are upregulated or downregulated in the presence of DMSP compared to the control conditions, respectively. The coloring of genes in 11A matches the coloring of genes in FIG. 10A.

FIG. 12 includes a non-limiting scheme and graphs showing the metabolic fate of DMSP methyl groups during the lag phase. The bacterium P. inhibens was treated with ¹³C-labeled DMSP, which resulted in the formation of ¹³C-labeled SAM and MTA after 2 hours of the lag phase. The result confirms that DMSP methyl groups are assimilated via the methionine cycle, resulting in the formation of SAM. SAM is required for synthesizing the polyamine spermidine, which generates MTA as side-product. The formation of labelled MTA, and the high transcription levels found for genes 83, 1 and 2 (FIGS. 10A and 11A), show that DMSP methyl groups are used for polyamine synthesis during the lag phase.

FIG. 13 includes a micrograph showing a 10% SDS-PAGE separation of the recombinant Bmt protein from P. inhibens and the MmuM methionine synthase protein from E. coli (1 μg). Both proteins were purified by affinity chromatography.

FIG. 14 includes a graph showing that Bmt synthesizes methionine using DMSP as methyl group donor. Methionine production detected from in vitro reactions containing purified Bmt enzyme together with the methyl group acceptor homocysteine (Hcy), and the methyl group donors DMSP (blue bar) or betaine (orange bar). A reaction containing purified methionine synthase MmuM from E. coli together with the substrates Hcy and S-methyl-L-methionine (SMM) was used as positive control (green bar). Results were compared to negative control reactions containing only DMSP and Hcy, Bmt and DMSP, or Bmt and Hcy (grey bars). Lines indicate the standard deviation of three different experiments.

FIGS. 15A-15B include graphs showing that Bmt is a methionine synthase required for lag phase shortening. (15A) The Δbmt mutant has impaired growth in minimal media (light gray dots) which is restored upon media supplementation with 200 μM methionine (dark gray dots). In both conditions, with (dark blue dots) and without (light blue dots) methionine, the lag phase duration is not influenced by 2 μM DMSP. (15B) The ΔmetE mutant growing in minimal media (light green dots) showed lag phase shortening in the presence of 2 μM DMSP (green dark dots).

FIG. 16 includes a graph showing that the co-factor tetrahydrofolate is not necessary for lag phase shortening. Cultures of P. inhibens growing with 50 ng of the Tetrahydrofolate inhibitor Trimethoprim (TMP) required the addition of 100u M dNTPs for growth. The further addition of 2M DMSP still induced lag phase shortening.

FIGS. 17A-17C include graphs showing that methylated compounds shorten the lag phase in other bacteria. The induction of the lag phase shortening by methylated compounds is also observed in (17A) the marine bacterium Vibrio sp. by 2 μM DMSP and betaine, (17B) the soil bacterium Bacillus subtillis by 2 μM DMSP and betaine, (17C) and the model bacterium E. coli BL21 by 2 μM S-methyl-L-methionine (SMM).

FIGS. 18A-18F include graphs showing that methylated compounds induce lag phase shortening in: Ruegeria sp. (18A), S. pontiacus (18B), P. agglomerans (18C), E. americana (18D), E. coli DH5a (18E), and E. coli K12 (18F).

FIGS. 19A-19B include vertical bar graphs showing that salt (19A) and temperature (19B) stresses enhance the lag phase shortening effect induced by a methyl group donor, such as DMSP.

### DETAILED DESCRIPTION OF THE INVENTION

According to one aspect, there is provided a transgenic or transformed microorganism heterologously expressing a polynucleotide encoding a betaine-homocysteine S-methyltransferase (Bmt).

In some embodiments, there is provided a composition comprising the transgenic or transformed microorganism as disclosed herein.

In some embodiments, the composition comprises a culture medium. In some embodiments, the culture medium is suitable for growth of the transgenic or transformed microorganism, as disclosed herein.

In some embodiments, the culture medium comprises compound(s) and/or element(s) suitable or required for growth of the transgenic or transformed microorganism, as disclosed herein.

Compounds and/or elements suitable or required for growth of microorganisms are common and would be apparent to one of ordinary skill in the art of microbiology. In some embodiments, the culture medium further comprises or is further supplemented with a methyl group donor as disclosed herein.

According to another aspect, there is provided a method for shortening lag phase of a microorganism. In some embodiments, the method comprises culturing a microorganism as disclosed herein.

In some embodiments, the method is not performed in a body, such as a human body. In some embodiments, contacting, subjecting, measuring, or any combination thereof, as disclosed herein, in not performed in a subject, such as a mammalian subject, e.g., a human subject.

In some embodiments, contacting, subjecting, measuring, or any combination thereof, as disclosed herein, in performed in a tube, plate, vessel, reactor, fermenter, or any equivalent thereof.

In some embodiments, the method is an in vitro method. In some embodiments, the method is performed in vitro. In some embodiments, in vitro is in a bottle, a vessel, a tube, or any equivalent thereof, know for a person of skill in the art of microbiology and cell biology as suitable for culturing microorganism(s). In some embodiments, in vitro culturing comprises fermentation, fermenting, being performed in a fermentor, or any combination thereof.

In some embodiments, the method comprises contacting the microorganism with an effective amount of a methyl group donor, thereby shortening lag phase of the microorganism.

In some embodiments, the method comprises contacting the microorganism with an effective amount of one-carbon group donor, thereby shortening lag phase of the microorganism.

As used herein, the term “methyl group donor” encompasses any compound comprising at least one methyl group and being capable of donating the methyl group to an acceptor or an accepting molecule.

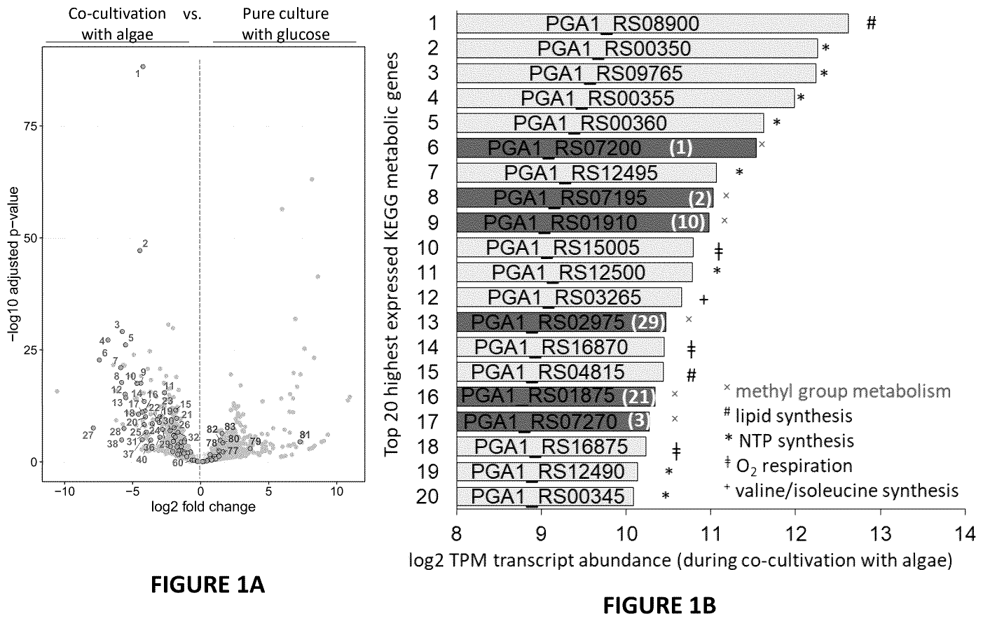

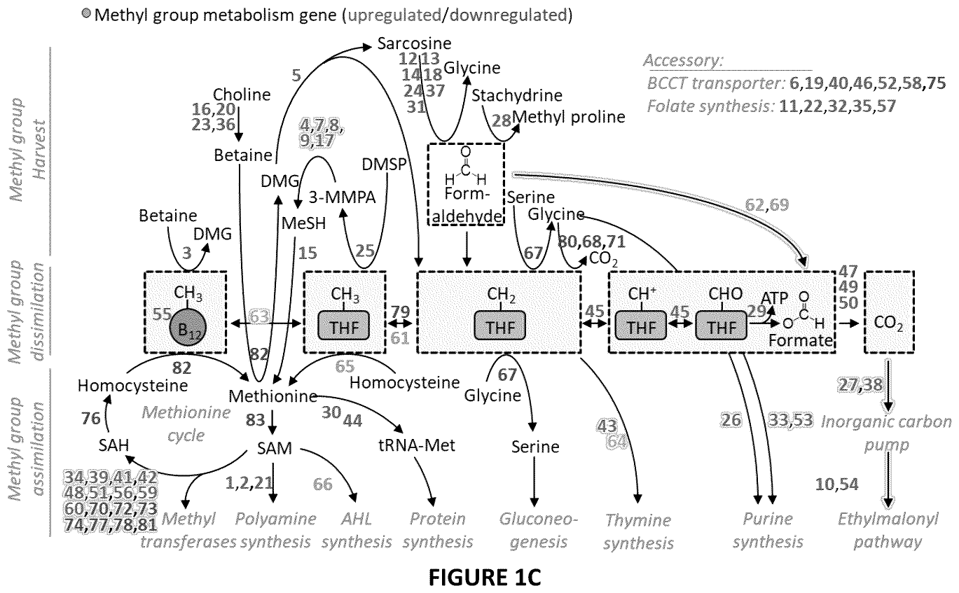

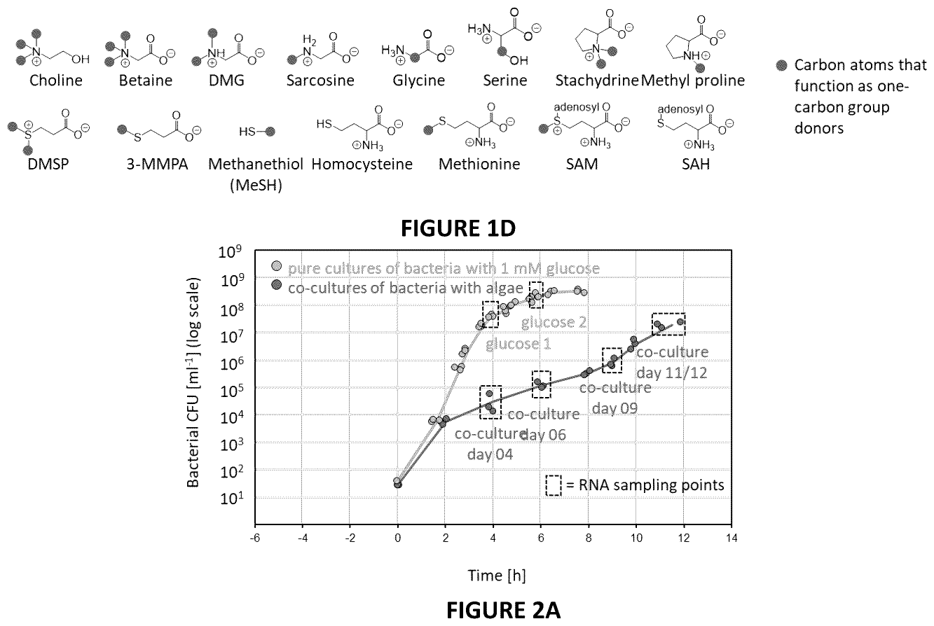

As used herein, the term “one-carbon group donor” encompasses any molecule that has a carbon atom, which can be channeled into, utilized, or donated to the “methyl group metabolism”, such as defined in FIG. 1C herein.

In some embodiments, one-carbon group donor comprises glycine, serine, an equivalent thereof, or any combination thereof.

In some embodiments, the acceptor or an accepting molecule comprises an amino acid or a precursor thereof.

In some embodiments, the acceptor or accepting molecule comprises a methionine precursor. In some embodiments, acceptor or an accepting molecule comprises homocysteine.

In some embodiments, a methyl group donor comprises a tertiary sulfonium group or a quaternary ammonium group. In some embodiments, a methyl group donor comprises a plurality of methyl group donors. In some embodiments, a plurality of methyl group donors comprises a plurality of types of methyl group donors. In some embodiments, a methyl group donor comprises a plurality of different types of methyl group donors, wherein each of the different types of methyl group donors comprises a different type of a quaternary ammonium group. In some embodiments, a methyl group donor comprises a plurality of different types of methyl group donors, wherein each of the different types of methyl group donors comprises a different type of a tertiary sulfonium group.

In some embodiments, a methyl group donor comprises a plurality of different types of methyl group donors, comprising at least one methyl group donor comprising a quaternary ammonium group and at least one methyl group donor comprising a tertiary sulfonium group.

In some embodiments, the methyl group donor comprises at least one methyl group.

In some embodiments, the methyl group donor comprises one or more methyl groups.

In some embodiments, the methyl group donor comprises a plurality of methyl groups.

As used herein, the term “plurality” comprises any integer equal to or greater than 2.

In some embodiments, the sulfonium group or a quaternary ammonium group comprises at least one, one or more, or a plurality of methyl groups.

In some embodiments, the one or more methyl groups is covalently bound to a sulfate/sulfur atom or a nitrogen atom.

In some embodiments, a methyl group donor comprises dimethylsulfoniopropionate (DMSP), betaine, choline, dimethylsulfonioacetate (DMSA), carnitine, homarine, stachydrine, trigonelline, gonyol, S-methylmethionine (SMM), or any combination thereof.

In some embodiments, a methyl group donor is selected from: glycine betaine, β-alanine betaine, proline betaine, hydroxyproline betaines, pipecolate betaine, choline O-sulfate, DMSP, trigonelline, acetylcholine, S-methyl-L-methionine, betaine aldehyde, γ-butyrobetaine, S-adenosyl-L-methionine, or any combination thereof.

In some embodiments, betaine is or comprises glycine betaine.

The terms “betaine” and “glycine betaine”, as used herein, are interchangeable.

In some embodiments, a methyl group donor comprises DMSP, betaine, choline, or any combination thereof. In some embodiments, a methyl group donor comprises (DMSP). In some embodiments, a methyl group donor comprises betaine. In some embodiments, a methyl group donor comprises choline. In some embodiments, a methyl group donor comprises DMSA. In some embodiments, a methyl group donor comprises carnitine. In some embodiments, a methyl group donor comprises homarine. In some embodiments, a methyl group donor comprises stachydrine. In some embodiments, a methyl group donor comprises trigonelline. In some embodiments, a methyl group donor comprises gonyol.

In some embodiments, a methyl group donor comprising a tertiary sulfonium group comprises DMSP, DMSA, gonyol, S-methyl-L-methionine, or any combination thereof.

In some embodiments, a methyl group donor comprising a quaternary ammonium group comprises: betaine, choline, homarine, carnitine, stachydrine, trigonelline, or any combination thereof.

In some embodiments, the method further comprises a step comprising supplementing or contacting the microorganism with an effective amount of: vitamin B12, folate, or both, and optionally with the methyl group donor disclosed herein.

In some embodiments, the method further comprises a step comprising subjecting the microorganism to a stress agent (or “a stressor”), or a plurality thereof.

In some embodiments, the step comprising subjecting the microorganism to a stress agent (or a stressor) or a plurality thereof, is performed before contacting the microorganism with an effective amount of a methyl group donor. In some embodiments, the step comprising subjecting the microorganism to a stress agent (or a stressor) or a plurality thereof, is performed after contacting the microorganism with an effective amount of a methyl group donor. In some embodiments, the step comprising subjecting the microorganism to a stress agent (or a stressor) or a plurality thereof, performed before and after contacting the microorganism with an effective amount of a methyl group donor.

In some embodiments, contacting the microorganism with an effective amount of a methyl group donor is performed under stress conditions.

In some embodiments, a stress agent is a biotic stress agent. In some embodiments, a stress agent is an abiotic stress agent. In some embodiments a stress agent comprises a plurality of stress agents. In some embodiments, a plurality of stress agents comprises one or more biotic stress agents. In some embodiments, a plurality of stress agents comprises one or more abiotic stress agents. In some embodiments, a plurality of stress agents comprises at least one biotic stress agent and at least one abiotic stress agent.

Non-limiting examples of stress agents include, but are not limited to, radiation, temperature, acidity, salinity, osmolarity, toxicity (such as, but not limited to, imposed by a toxin), aerobic/anaerobic conditions, shear force, etc.

In some embodiments, a stress agent or stressor comprises: temperature, pH, salinity, or any combination thereof.

In some embodiments, a stress agent or stressor comprises: suboptimal temperature, suboptimal pH, suboptimal salinity, or any combination thereof.

Methods for determining that a compound induced stress over a cell as disclosed herein, thus affect cell viability, survival, activity, performance, etc., are common and would be apparent to one of ordinary skill in the art of microbiology. Non-limiting examples for such methods include, but are not limited to, viability stain (e.g., trypan blue), spectrometry, FACS, MTT, XTT, and others.

The terms “stress agent” and “stressor” are used herein interchangeably.

In some embodiments, the method further comprises a step comprising subjecting the microorganism to suboptimal salt concentration, suboptimal temperature, or both.

In some embodiments, step comprising subjecting the microorganism to suboptimal salt concentration, suboptimal temperature, or both is performed before contacting the microorganism with an effective amount of a methyl group donor.

In some embodiments, step comprising subjecting the microorganism to suboptimal salt concentration, suboptimal temperature, or both is performed after contacting the microorganism with an effective amount of a methyl group donor.

In some embodiments, step comprising subjecting the microorganism to suboptimal salt concentration, suboptimal temperature, or both is performed before and after contacting the microorganism with an effective amount of a methyl group donor.

In some embodiments, contacting the microorganism with an effective amount of a methyl group donor is performed under suboptimal salt concentration, suboptimal temperature, or both.

In some embodiments, contacting the microorganism with an effective amount of a methyl group donor is performed under stress conditions. In some embodiments, contacting the microorganism with an effective amount of a methyl group donor is performed under suboptimal salt concentration, suboptimal temperature, or both.

In some embodiments, a suboptimal temperature is higher or greater than the optimal temperature which would be apparent to one of ordinary skill in the art, as being the suitable temperature for culturing the microorganism (e.g., a wild-type variant of the microorganism).

In some embodiments, a suboptimal salt concentration is higher or greater than the optimal salt concentration which would be apparent to one of ordinary skill in the art, as being the suitable salt concentration for culturing the microorganism (e.g., a wild-type variant of the microorganism).

In some embodiments, a suboptimal temperature is lower than the optimal temperature which would be apparent to one of ordinary skill in the art, as being the suitable temperature for culturing the microorganism (e.g., a wild-type variant of the microorganism).

In some embodiments, a suboptimal salt concentration is lower than the optimal salt concentration which would be apparent to one of ordinary skill in the art, as being the suitable salt concentration for culturing the microorganism (e.g., a wild-type variant of the microorganism).

In some embodiments, the microorganism is a transgenic, transformed, or transduced cell.

In some embodiments, a transgenic, transformed, or transduced cell is cultured under effective conditions, which allow for the expression of high amounts of a recombinant polypeptide. In some embodiments, effective culture conditions include, but are not limited to, effective media, bioreactor, temperature, pH and oxygen conditions that permit protein production. In one embodiment, an effective medium refers to any medium in which a cell is cultured to produce the recombinant polypeptide of the present invention. In some embodiments, a medium typically includes an aqueous solution having assimilable carbon, nitrogen and phosphate sources, and appropriate salts, minerals, metals and other nutrients, such as vitamins. In some embodiments, cells of the present invention can be cultured in conventional fermentation bioreactors, shake flasks, test tubes, microtiter dishes and petri plates. In some embodiments, culturing is carried out at a temperature, pH and oxygen content appropriate for a recombinant cell. In some embodiments, culturing conditions are within the expertise of one of ordinary skill in the art.

In some embodiments, the transgenic cell or transformed cell heterologously expresses a polynucleotide encoding a betaine-homocysteine S-methyltransferase (Bmt) polypeptide.

The terms “polypeptide”, “peptide” and “protein” are used herein interchangeably and refer to a polymer of amino acid residues. In another embodiment, the terms “polypeptide”, “peptide” and “protein” as used herein encompass native peptides, peptidomimetics (typically including non-peptide bonds or other synthetic modifications) and the peptide analogues peptoids and semipeptoids or any combination thereof. In one embodiment, any one of the terms “polypeptide”, “peptide” and “protein” applies to naturally occurring amino acid polymers. In another embodiment, any one of the terms “polypeptide”, “peptide” and “protein” applies to amino acid polymers in which one or more amino acid residue is an artificial chemical analogue of a corresponding naturally occurring amino acid.

In some embodiments, the polynucleotide encoding a Bmt is codon optimized for expression in a microorganism as disclosed herein.

As used herein, the term “codon optimized” describes a sequence that encodes identical amino acids to those encoded by a non-optimized codon sequence (synonymous codon), however, at least one of: translation rate of the codon optimized sequence, protein product amount, duration of protein structure stability, or any combination thereof, is increased, compared to the non-optimized codon. An ordinary skill in the art will know how to optimize a codon sequence for its expression in the desired cell, using a codon optimization gene engineering tool, comprising, but not limited to, algorithms that analyze codon optimization based on the codon frequencies in the desired cell/species (e.g., ‘codon preference’). In some embodiments, increased one of: translation rate, protein product amount, and duration of structure stability, is by at least by 30%, compared to a control, such as, a polynucleotide comprising a non-codon optimized sequence.

In some embodiments, the polynucleotide encoding a Bmt is operably linked to a promoter. In some embodiments, the promoter is an induced promoter or a constitutive promoter.

In some embodiments, the polynucleotide encoding a Bmt is overexpressed under the regulation of an induced promoter or a constitutive promoter.

Types of promoters as mentioned herein are common and would be apparent to one of ordinary skill in the art of molecular biology and microbiology.

In some embodiments, the promoter comprises a bacterial promoter. In some embodiments, the bacterial promoter comprises a promoter known to induce expression or overexpression of a gene in a bacterial cell.

In some embodiments, a microorganism is selected from: bacterium, fungus, a microalga, or any combination thereof.

In some embodiments, a fungus comprises a yeast.

In some embodiments, a bacterium comprises: P. inhibens, E. coli, B. subtilis, or any combination thereof.

Expressing a gene within a cell is well known to one skilled in the art. It can be carried out by, among many methods, transfection, viral infection, or direct alteration of the cell's genome. In some embodiments, the gene is in an expression vector such as plasmid or viral vector. One such example of an expression vector containing p16-Ink4a is the mammalian expression vector pCMV p16 INK4A available from Addgene.

A vector nucleic acid sequence generally contains at least an origin of replication for propagation in a cell and optionally additional elements, such as for a heterologous expression of a polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoters may be a viral promoter.

In some embodiments, the genet, e.g., as disclosed herein, is operably linked to a promoter. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell).

In some embodiments, the vector is introduced into the cell by standard methods including electroporation (e.g., as described in From et al., Proc. Natl. Acad. Sci. USA 82, 5824 (1985)), heat shock, infection by viral vectors, high velocity ballistic penetration by small particles with the nucleic acid either within the matrix of small beads or particles, or on the surface (Klein et al., Nature 327. 70-73 (1987)), and/or the like.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (+), pGL3, pZcoSV2 (+), pSccTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-IMTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In some embodiments, the method further comprises a step preceding the contacting, comprising transfecting or transforming a microorganism with a polynucleotide encoding a Bmt transcript or protein product thereof.

In some embodiments, the polynucleotide comprises a nucleic acid sequence: ATGACAAACACTTTCACCACCCTGCTGGAGACCAAAGACGCCCTGCTTGCGGATGGGGCC ACCGGCACCAACCTGTTCAACATGGGCCTCCAGTCCGGTGATGCGCCGGAGCTGTGGAAT GTGGATGAACCCAAGAAAATCACCGCGCTCTATCAGGGGGCGGTCGATGCGGGCAGCGA TCTGTTCCTGACCAATACCTTTGGCGGGACCGCCGCGCGGCTGAAGCTGCACGACGCCCA CCGCCGGGTCCGGGAGCTGAACGTCGCGGGGGCCGAGTTGGGCCGCAACGTCGCGGATC GCTCTGAGCGCAAGATCGCCGTGGCCGGATCAGTCGGACCGACTGGCGAAATCATGCAG CCGGTGGGTGAACTGAGCCACGCGCTCGCCGTGGAAATGTTCCATGAGCAGGCCGAGGC GCTGAAAGAGGGCGGCGTCGACGTGTTGTGGCTGGAGACGATCTCTGCTCCGGAAGAGT ACCGCGCCGCCGCTGAAGCGTTCAAACTGGCGGATATGCCCTGGTGCGGCACCATGAGTT TTGACACCGCCGGGCGCACCATGATGGGGGTCACCTCCGCCGATATGGCGCAGCTGGTCG AGGAGTTCGACCCAGCGCCTCTGGCCTTTGGTGCCAATTGCGGCACCGGGGCGTCCGACA TTCTGCGCACGGTACTTGGGTTCGCCGCCCAGGGCACGACCCGCCCGATCATTTCCAAGG GCAATGCCGGGATCCCGAAATATGTCGATGGTCATATCCACTATGACGGCACGCCGACCC TGATGGGGGAATATGCAGCCATGGCAAGAGATTGCGGCGCCAAAATCATTGGTGGCTGCT GTGGCACCATGCCGGATCACCTGCGCGCCATGCGCGAGGCGCTGGATACCCGCCCCCGGG GCGAGCAGCTAACACTGGAGCGGATCGTTGAGGTGCTTGGTCCCTTCACCTCCGACAGTG ACGGCACCGGTGAGGATACAGCCCCTGACCGCCGCAGCCGTCGCGGTCGCCGTCGCGGCT GA (SEQ ID NO): 1, or a functional analog thereof having at least 80% sequence homology or identity thereto.

In some embodiments, a Bmt polypeptide encoded from the polynucleotide disclosed herein comprises the amino acid sequence: MTNTFTTLLETKDALLADGATGTNLFNMGLQSGDAPELWNVDEPKKITALYQGAVDAGSDL FLTNTFGGTAARLKLHDAHRRVRELNVAGAELGRNVADRSERKIAVAGSVGPTGEIMQPVGE LSHALAVEMFHEQAEALKEGGVDVLWLETISAPEEYRAAAEAFKLADMPWCGTMSFDTAGR TMMGVTSADMAQLVEEFDPAPLAFGANCGTGASDILRTVLGFAAQGTTRPIISKGNAGIPKYV DGHIHYDGTPTLMGEYAAMARDCGAKIIGGCCGTMPDHLRAMREALDTRPRGEQLTLERIVE VLGPFTSDSDGTGEDTAPDRRSRRGRRRG (SEQ ID NO: 2), or a functional analog thereof having at least 80% sequence homology or identity thereto.

The terms “polynucleotide”, “polynucleotide sequence”, “nucleic acid sequence”, and “nucleic acid molecule” are used interchangeably herein. These terms encompass nucleotide sequences and the like. A polynucleotide may be a polymer of RNA or DNA that is single- or double-stranded, that optionally contains synthetic, non-natural or altered nucleotide bases.

The term “functional analog” as used herein, generally refers to any polynucleotide encoding a peptide characterized by having betaine-homocysteine S-methyltransferase activity or functionality, as disclosed herein. The term “functional analog” as used herein, generally refers to any polypeptide, peptide, or protein characterized by having betaine-homocysteine S-methyltransferase activity or functionality, as disclosed herein.

In some embodiments, a functional analog as disclosed herein has at least 50%, 60%, 70%, 80%, 90%, 95%, or 99% sequence homology or identity to SEQ ID NO: 1 or SEQ ID NO: 2, or any value and range therebetween. Each possibility represents a separate embodiment of the invention. In some embodiments, a functional analog has at least 50-100%, 60-100%, 70-100%, 80-100%, or 90-100% sequence homology or identity to SEQ ID NO: 1 or SEQ ID NO: 2. Each possibility represents a separate embodiment of the invention.

In some embodiments, a sequence is a nucleic acid sequence. In some embodiments, a sequence is an amino acid sequence.

According to another aspect, there is provided a composition comprising the transgenic cell or transformed cell disclosed herein. In some embodiments, the composition further comprises a carrier or an excipient. In some embodiments, the carrier or an excipient is a biologically acceptable carrier or an excipient.

As used herein, the term “carrier”, “excipient”, or “adjuvant” refers to any component of a composition that is not “the active agent”, e.g., the cell. Any non-toxic, inert, and effective carrier may be used to formulate the compositions contemplated herein. The carrier may comprise, in total, from about 0.1% to about 99.99999% by weight of the compositions presented herein.

The terms, “microorganism” and “cell” as used herein, are interchangeable.

In some embodiments, the microorganism is a unicellular microorganism.

According to another aspect, there is provided a method of screening for a compound being suitable for shortening the lag phase of a microorganism. In some embodiments, the method comprises screening for a compound being suitable for shortening the lag phase of a microorganism.

In some embodiments, the method comprises contacting a microorganism with compound and measuring the length of a lag phase of the microorganism or a culture comprising same in the presence of the compound.

In some embodiments, a reduction in the length lag phase of the microorganism or a culture comprising thereof in the presence of the compound compared to the length lag phase of the microorganism or a culture comprising thereof in the absence of the compound is indicative that the compound is suitable for shortening the lag phase of a microorganism.

In some embodiments, maintenance of or prolongation of the length lag phase of the microorganism or a culture comprising thereof in the presence of the compound compared to the length lag phase of the microorganism or a culture comprising thereof in the absence of the compound is indicative that the compound is unsuitable for shortening the lag phase of a microorganism. In some embodiments, the compound is a methyl group donor.

### General

Any number range recited herein relating to any physical feature, such as sequence homology or identity, are to be understood to include any integer within the recited range, unless otherwise indicated.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

About refers to ±10%.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Additional objects, advantages, and novel features of the present invention will become apparent to one ordinarily skilled in the art upon examination of the following examples, which are not intended to be limiting. Additionally, each of the various embodiments and aspects of the present invention as delineated herein above and as claimed in the claims section below finds experimental support in the following examples.

### EXAMPLES

Generally, the nomenclature used herein, and the laboratory procedures utilized in the present invention include molecular, biochemical, bioengineering, bioprocessing, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8^{th} Edition), Appleton & Lange, Norwalk, CT (1994); Mishell and Shiigi (eds), “Selected Methods in Cellular Immunology”, W. H. Freeman and Co., New York (1980); Molecular Cell Biology Berk A. et al. 8^{th} edition; Molecular Biotechnology: Principles and Applications of Recombinant DN, Glick BR. 5^{th} edition; Culture of Animal Cells: A Manual of Basic Technique and Specialized Applications Freshney IR, 7^{th} edition; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., cd. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference. Other general references are provided throughout this document.

### Materials and Methods

### Strains and Culture Conditions

The bacterial strain Phaeobacter inhibens DSM 17395 was purchased from the German collection of microorganisms and cell cultures (DSMZ, Braunschweig, Germany) and stored at −80° C. with 20% glycerol. Routine cultivation of P. inhibens was conducted at 30° C. in artificial seawater medium (ASW) based on Goyet and Poisson (1989). The ASW medium contained mineral salts (NaCl, 409.41 mM; Na₂SO₄, 28.22 mM; KCl, 9.08 mM; KBr, 0.82 mM; NaF, 0.07 mM; Na₂CO₃, 0.20 mM; NaHCO₃, 2 mM; MgCl₂, 50.66 mM; SrCl₂, 0.09 mM), L1 vitamins (thiamine HCl, 100 μg/L; biotin, 0.5 μg/L; vitamin B₁₂, 0.5 g/L), L1 trace elements (Na₂EDTA·2H₂O, 4.36 mg/L; FeCl₃·6H₂O, 3.15 mg/L; MnCl₂·4H₂O, 178.1 μg/L; ZnSO₄·7H₂O, 23.0 μg/L; CoCl₂·6H₂O, 11.9 μg/L; CuSO₄·5H₂O, 2.5 μg/L; Na₂MoO₄·2H₂O, 19.9 μg/L; H₂SeO₃, 1.29 μg/L; NiSO₄·6H₂O, 2.63 μg/L; Na₃ VO₄, 1.84 μg/L; K₂CrO₄, 1.94 μg/L), and L1 nutrients (NaNO₃, 882 μM; NaH₂PO₄, 36.22 μM). ASW was adjusted to pH 8 using HCl. If not otherwise stated, the medium was further supplemented with a source of carbon (glucose, 1 mM), nitrogen (NH₄Cl, 5 mM), and sulfur (Na₂SO₄, 33 mM), referred to as ASW+CNS. Methylated effector compounds (e.g., donors) were administered at 1 or 2 μM in routine assays, or in higher concentrations as stated. The bacterium Bacillus subtilis strain 168 was cultivated in M9 Medium (Na₂HPO₄, 6 g/L; KH₂PO₄, 3 g/L; NaCl, 0.5 g/L; NH₄CL, 1 g/L; CaCl₂), 100 M; MgSO₄, 1 mM; Thiamine, 5 mg/L; EDTA, 50 mg/L; FcCl₃, 4.98 mg/L; ZnCl₂, 0.84 mg/L; CuCl₂·2H₂O, 0.13 mg/L; CoCl₂·6H₂O, 0.1 mg/L; H₃BO₃, 0.1 mg/L; MnCl₂·4H₂O, 0.016 mg/L; pH 7.1) with 20 mM glucose. Other bacterial strains tested for lag phase shortening were isolated from the marine environment (Despotović et al., 2021), and cultivated in ASW+CNS. A 96-well Tecan Infinite M Plex plate reader was used for high-resolution monitoring of bacterial growth dynamics at 30° C. An ImageStreamX Flow Imager system (Amnis) was used to characterize bacterial populations during the lag phase (FIG. 6). The growth of bacteria in co-culture with algae was monitored by counting colony forming units (CFU) on ½ YTSS agar plates. The growth of algae was monitored with a CellStream flow cytometer (Merck, Darmstadt, Germany).

### Dual RNA-Sequencing

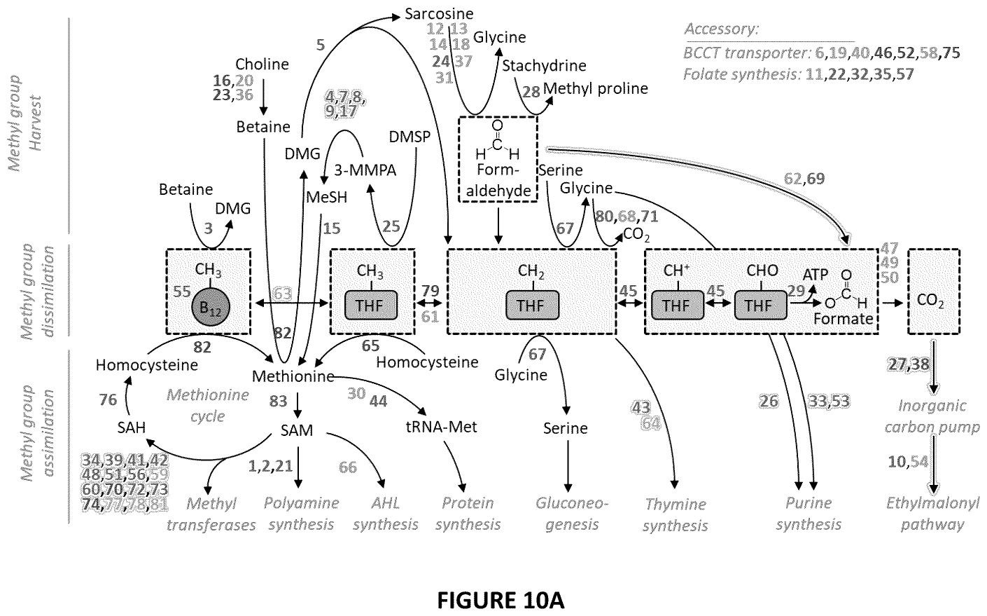

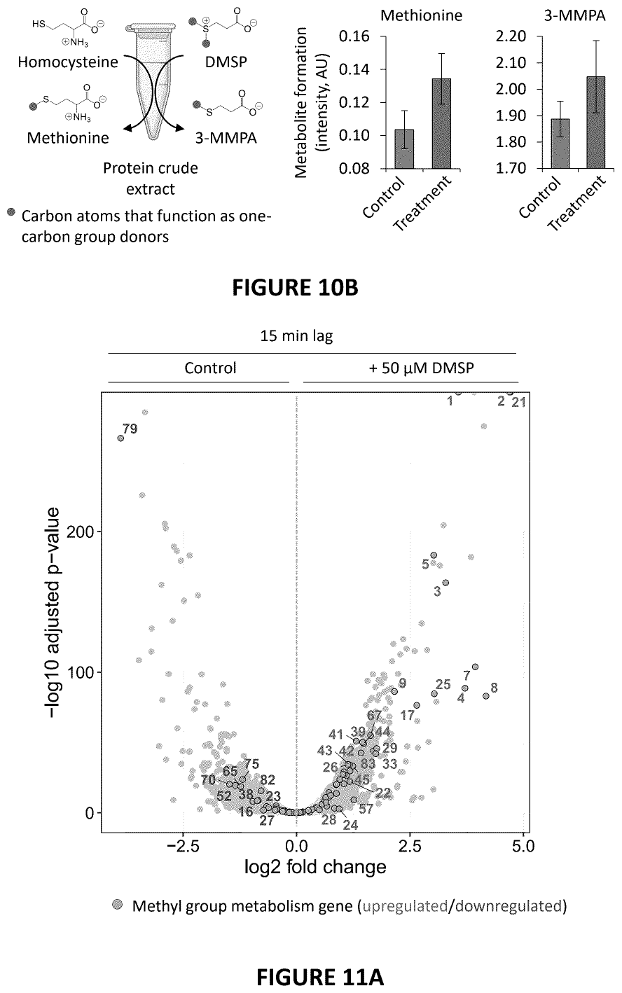

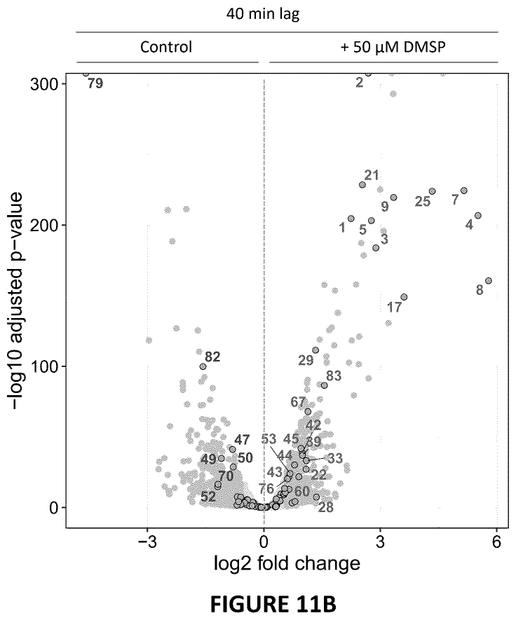

The bacterium P. inhibens DSM 17395 was co-cultivated with the alga Emiliania huxleyi CCMP3266 (purchased from Bigelow Laboratory for Ocean Sciences, East Boothbay, ME) to investigate the metabolic response of the bacterium in the presence of algae, compared to bacteria grown as pure culture with 2 mM glucose. The analysis was conducted using a dual RNA-Sequencing method adapted from Avraham et al. 2016. The adapted method was previously described by Sperfeld et al., 2021. Differentially upregulated and downregulated genes were identified by using DESeq2 (false discovery rate adjusted p-value<0.1; log 2 fold change>0.585). The resulting gene expression data, together with data from public databases (BioCyc and KEGG), were used to reconstruct the methyl group metabolism in P. inhibens DSM 17395 (FIG. 1C). The same dual RNA-sequencing method was used to identify genes that are differentially expressed during the lag phase of P. inhibens pure cultures treated with 50 μM DMSP, compared to control cultures without added DMSP (FIGS. 10A, and 11A-11B).

Methionine Synthesis Activity Assay with P. inhibens Protein Crude Extracts

The bacterium P. inhibens DSM 17395 was cultivated in 100 mL ASW with 5.5 mM glucose until reaching stationary phase, and then treated with 1 μM DMSP for 2 hours. Cell pellets of control and treated bacteria were harvested by 10 min centrifugation (15,000 rpm) at 4° C. The pellets were washed with buffer A (50 mM Hepes-KOH PH 7.5), resuspended in buffer B (50 mM Hepes-KOH PH 7.5, 10 mM β-mercaptoethanol, 1 mM EDTA) and disrupted by 5 min bead beating at 30 s⁻¹ with 300 mg silica beads (Mixer Mill MM-400, Retsch, Haan, Germany). The supernatant was collected by 5 min centrifugation (15,000 rpm). To measure the activity of enzymatic DMSP demethylation and methionine formation, 0.5 mL crude extract (1 mg/mL protein; determined with Bio-Rad protein assay, Hercules, California, U.S.) were incubated for 1 h with 1 mL Buffer C (60 mM PPB, 4.5 mM homocysteine, 4.5 mM DMSP, pH 7.5). The reaction mix was snap frozen in liquid nitrogen, and subsequently analyzed for product formation using LC/MS (FIG. 10).

Lag Phase Metabolomics with ¹³C-Labeled DMSP

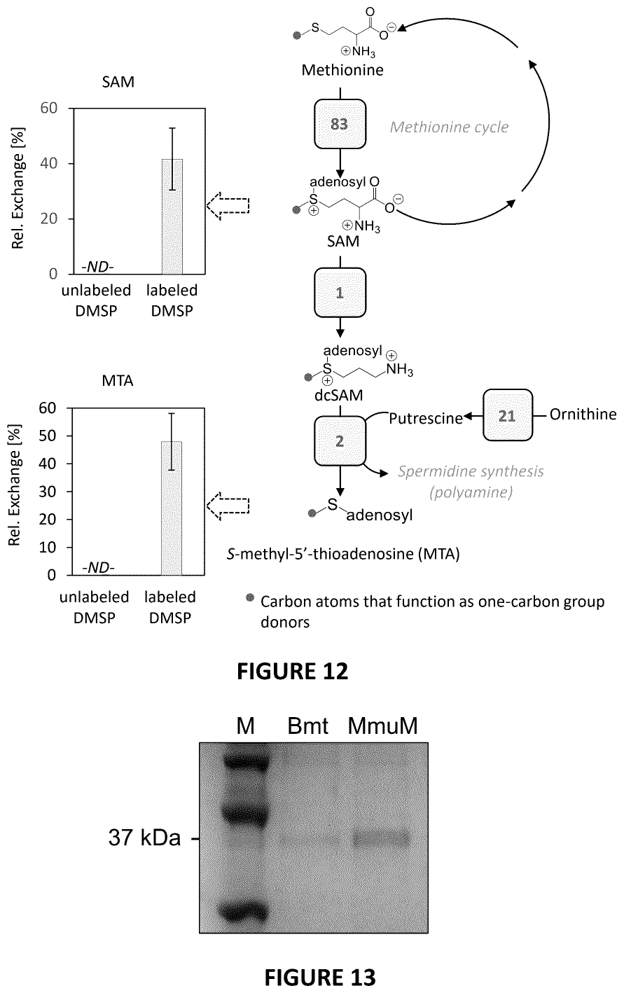

DMSP was synthesized in which both S-methyl groups are ¹³C-labeled, using the protocol of Wirth and Whitman (2018). Freshly initiated P. inhibens cultures (OD 0.01, ASW+CNS medium) were treated with 50 μM 13C-labeled DMSP and compared to reference cultures with non-labeled DMSP. Bacterial cells were harvested and extracted two hours after adding DMSP. The incorporation of the 13C label was analyzed by LC/MS (FIG. 12).

P. inhibens Knockout Mutants

For creation of P. inhibens knockout cells, ˜1,000 bp regions upstream and downstream of the gene of interest were amplified by PCR (Phusion High-Fidelity DNA polymerase, Thermo Scientific), using primers that added homologous regions to gentamycin or kanamycin resistance markers. The fragments were assembled and cloned into the TOPOII vector (Invitrogen) using restriction-free cloning (Peleg Y. & Unger T., 2014), generating a knockout (KO) plasmid. P. inhibens electrocompetent cells (300 μl) were transformed with 10 μg of the constructed KO plasmid by a pulse of 1.8 kV (Bio Rad). Cells were selected on ½YTSS agar plates containing 30 μg/ml gentamycin or 150 μg/ml kanamycin. Successful knockouts in single cell clones were verified by PCR and sanger sequencing.

### Protein Purification and Methionine Synthesis In Vitro Assay

The expression plasmid pET29b encoding the bmt gene with a Strep-tag II peptide sequence fused to the N-terminus was purchased from Twist Biosciences. The methionine synthase gene mmuM from E. coli was PCR-amplified (Phusion High-Fidelity DNA polymerase, Thermo Scientific) and cloned into the pET29b vector using the CPEC technique (Quan J. & Tian J., 2009). Resulting clones were validated by sanger sequencing. E. coli BL21 electrocompetent cells were transformed with 100 μg of the expression vectors, and cells were selected on LB agar plates containing 50 μg/ml kanamycin. Bacteria were grown in Tryptone Yeast extract Glucose (TYG) medium supplemented with 50 μg/ml kanamycin at 37° C. to mid-log phase (OD₆₀₀ 0.7). Then, cultures were induced with 0.2 mM of IPTG for 3 hours at 37° C. Bacteria were harvested by centrifugation (4,000 rpm for 15 min at 4° C.) and cells were resuspended in 20 mL of NP buffer (50 mM NaH₂PO₄, 300 mM NaCl, pH 8) and 100 μl of 10× Protease inhibitor (Sigma). Resuspended cells were filtered with a Miracloth (Sigma) and passed three times through a French Pressure cell (15,000 psi) for disruption. Bacterial lysates were clarified by centrifugation at 4,000 rpm for 15 min at 4° C. The supernatant was loaded onto a column containing Strep-tag II beads (IBA-Lifesciences) and then eluted following the manufacturer's recommendations. Proteins were concentrated (using Spin-X filters with 10 kDa cutoff, Corning) in 20 mM Hepes-KOH buffer pH 7.5. The Protein concentration was determined with the Protein Assay Dye Reagent Concentrate (BioRad) following the manufacturer's instructions. One microgram protein was resuspended in SDS-sample buffer and ran on a 10% SDS-PAGE (FIG. 13).

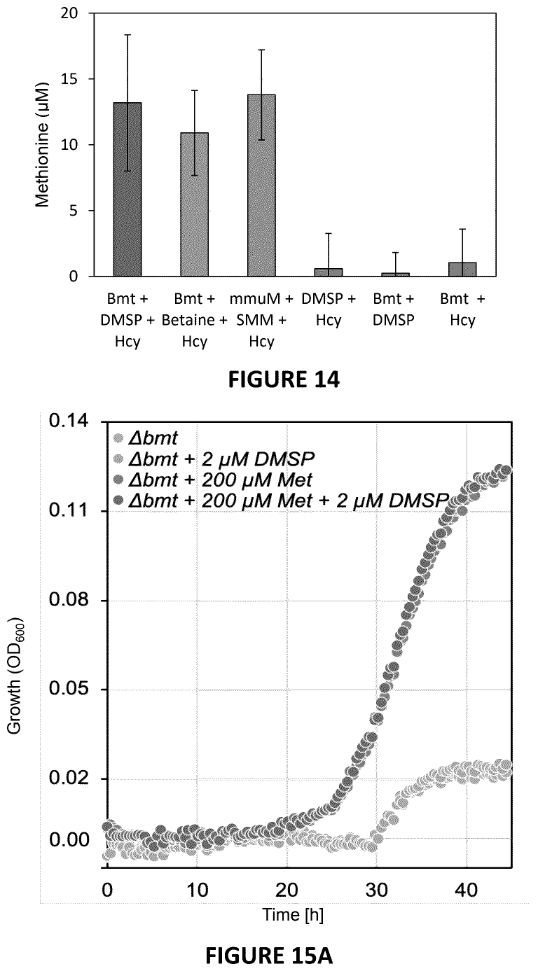

The methionine synthesis in vitro assay was based on the protocol of Ranocha et al., 2000. Reactions containing 20 mM Hepes-KOH buffer pH 7.5, 2 mM DTT, 2 mM homocysteine (Hcy), 200 μM methyl group donor (DMSP or betaine) and 200 μM pure Bmt were incubated at 30° C. for 2 h (final volume 50 μl). Methionine formation was measured from 20 μl of the in vitro reactions using the Fluorometric Methionine Assay Kit (Sigma; results depicted in FIG. 14).

### Example 1

### Co-Cultivation with Algae Elicits Bacterial Genes Involved in Methyl Group Metabolism

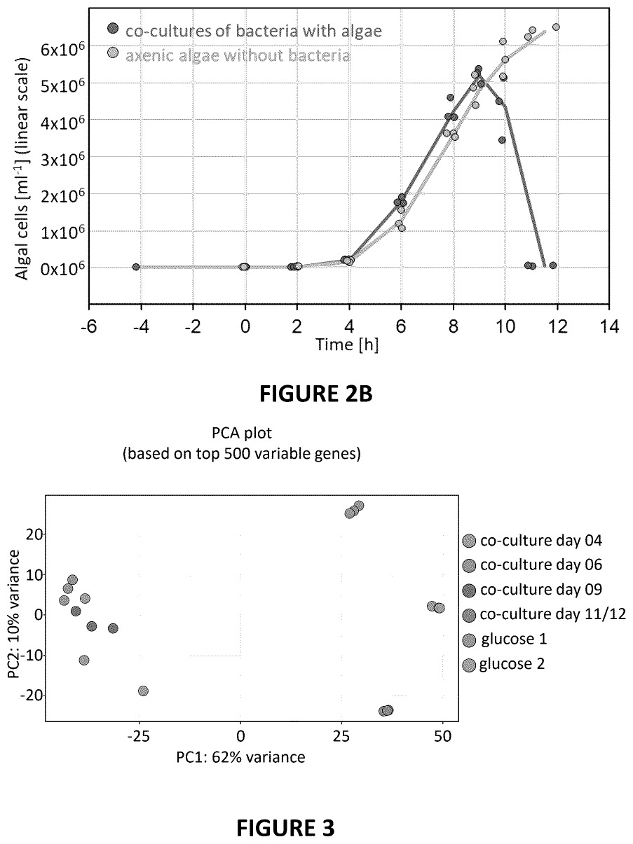

The inventors first aimed to identify the metabolic response and the underlying genes that are elicited in the bacterium P. inhibens during co-cultivation with the microalga Emiliania huxleyi. This algal-bacterial pair is environmentally relevant and was previously studied by the current inventors and others. To gain insight into the bacterial response towards microalgae, the inventors performed transcriptomic analysis of bacteria during co-cultivation with algae; a condition under which the bacteria rely exclusively on algal secreted metabolites for growth. The data were compared with the transcriptome of bacteria cultivated in pure culture with glucose as a carbon source (FIGS. 1-3). The inventors found that bacterial genes involved in methyl group metabolism, also termed “one-carbon metabolism”, were highly upregulated in the presence of algae (FIG. 1A). Methyl group metabolism genes were among the highest expressed metabolic genes, and their expression levels were as high as for genes involved in crucial metabolic functions such as oxygen respiration and ATP synthesis (FIG. 1B). To map the possible routes in which methyl groups are metabolized in the bacterial cell, the inventors reconstructed the methyl group metabolism pathway of P. inhibens (FIG. 1C). Overlaying the current transcriptomics data onto the reconstructed pathway indicated upregulation of reactions required for harvesting methyl groups from donor compounds, dissimilatory reactions involved in transforming methyl groups into ATP, and assimilatory reactions required for utilizing methyl groups as building blocks. Thus, the current transcriptome data revealed that methyl group metabolism is activated in bacteria during co-cultivation with algae, and that methyl groups are shuttled into both dissimilatory and assimilatory branches of the pathways.

### Example 2

### The Methylated Compound DMSP Shortens the Lag Phase of Bacteria

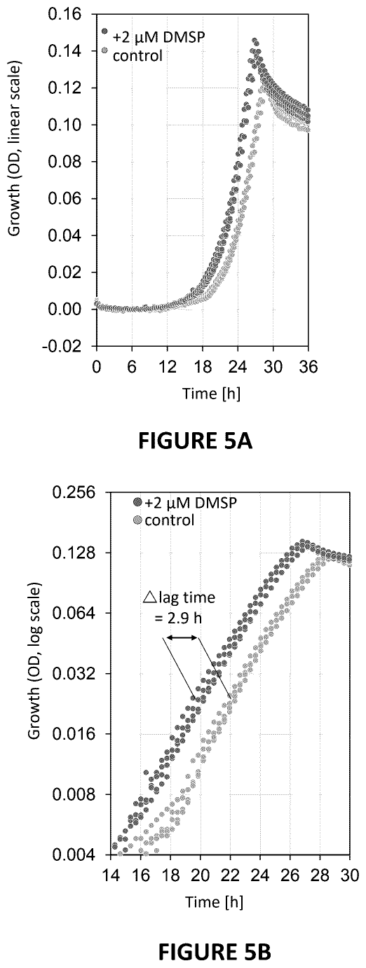

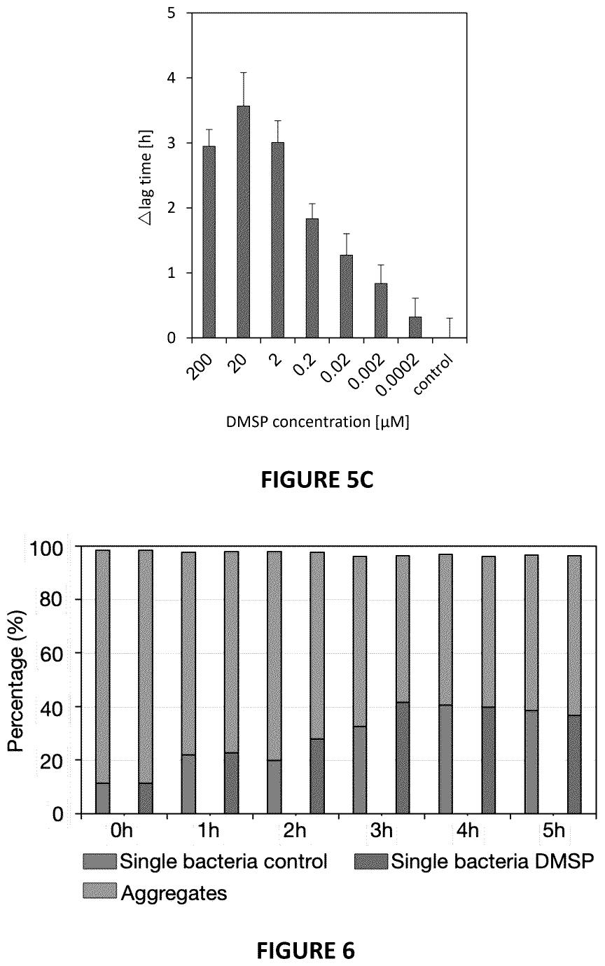

To test whether methylated compounds affect the growth of bacteria, the inventors cultivated bacterial pure cultures with glucose as main growth substrate and supplemented the medium with the well-studied methylated compound dimethylsulfoniopropionate (DMSP). DMSP that is naturally produced by microalgae and found in detectable levels in seawater, is known to be metabolized by various bacteria. The inventors found that micromolar concentrations of DMSP stimulate the growth of the bacterium (FIG. 5A). To characterize the stimulatory effect, the inventors plotted the growth curve on a semi-logarithmic scale, which showed that the lag time was shortened, while the growth rate was not affected (FIG. 5B). To quantify the lag phase shortening effect, the inventors calculated the Δlag time, which is the time difference between the commence of exponential growth in control cultures compared to treated cultures (FIG. 5B). The inventors analyzed Δlag times under different DMSP concentrations and found that already nanomolar levels of DMSP induce a significant lag phase shortening (FIG. 5C).

To reflect the natural low bacterial abundance in the oligotrophic ocean with low microalgal productivity, the current bacterial growth experiments were routinely initiated with an optical density (OD) of 0.00001. Such low initial ODs challenge the detection of cell divisions that occur prior to reaching an OD of at least 0.001, which is the theoretical limit of detection. To determine the actual length of the lag phase, the inventors analyzed bacterial cultures using imagining flow cytometry. Based on this method, the bacterial lag phase duration is about four hours (FIG. 6).

### Example 3

Methyl groups are involved in lag phase shortening

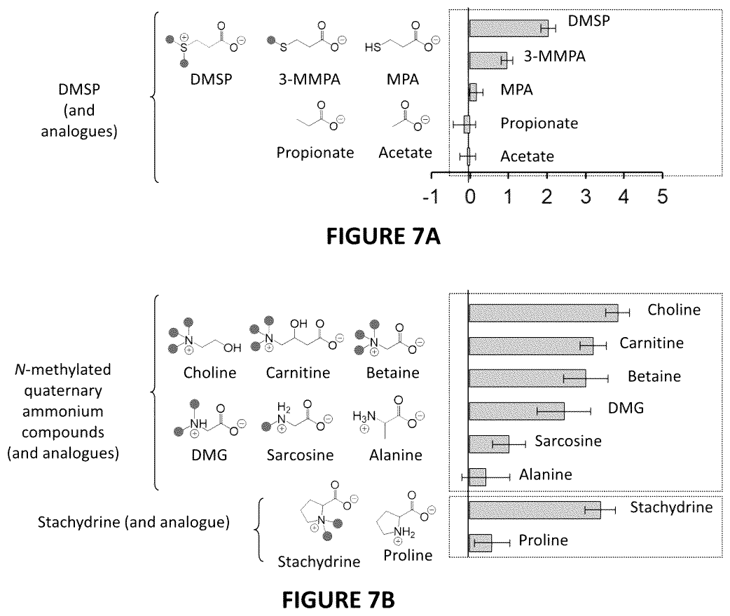

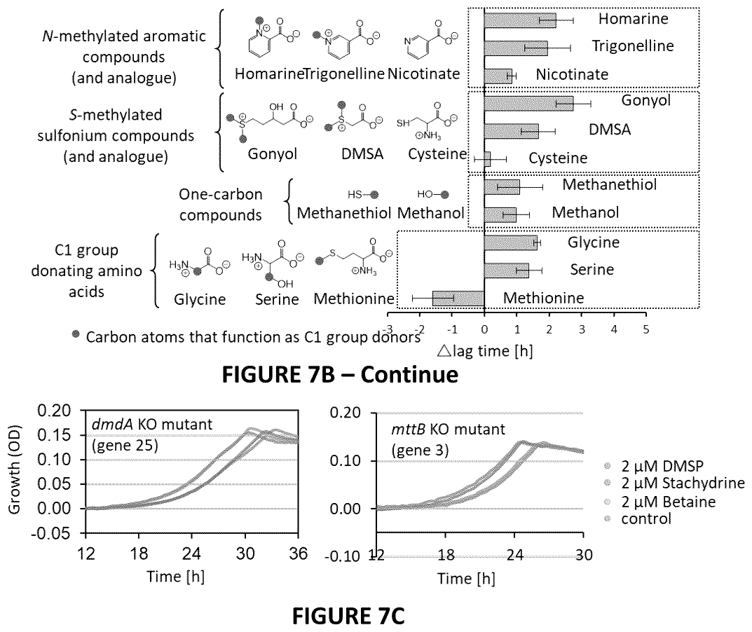

Next, the inventors inquired whether lag phase shortening is caused by the methyl groups of DMSP, or whether other parts of the DMSP molecule, such as the propionate backbone and the reduced sulfur group, are involved. To test this, the inventors measured the Δlag time under treatment with analogues of DMSP that harbor different amounts of methyl groups (FIG. 7A). Lag phase shortening was only observed for analogues that carry methyl groups, and the extent of lag phase shortening was found to correlate with the number of methyl groups. These data highlight that DMSP methyl groups are involved in lag phase shortening.

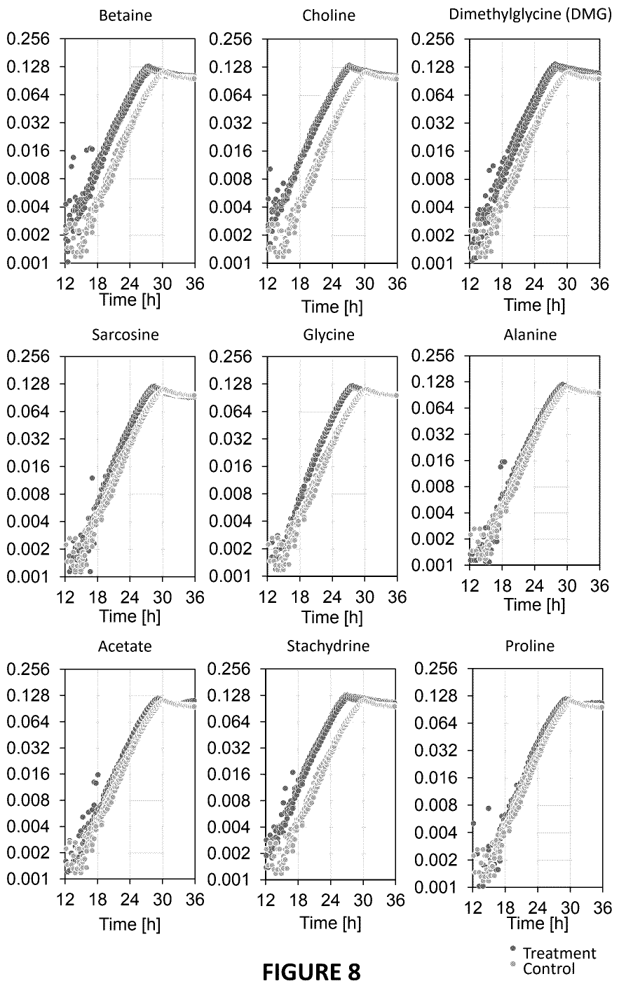

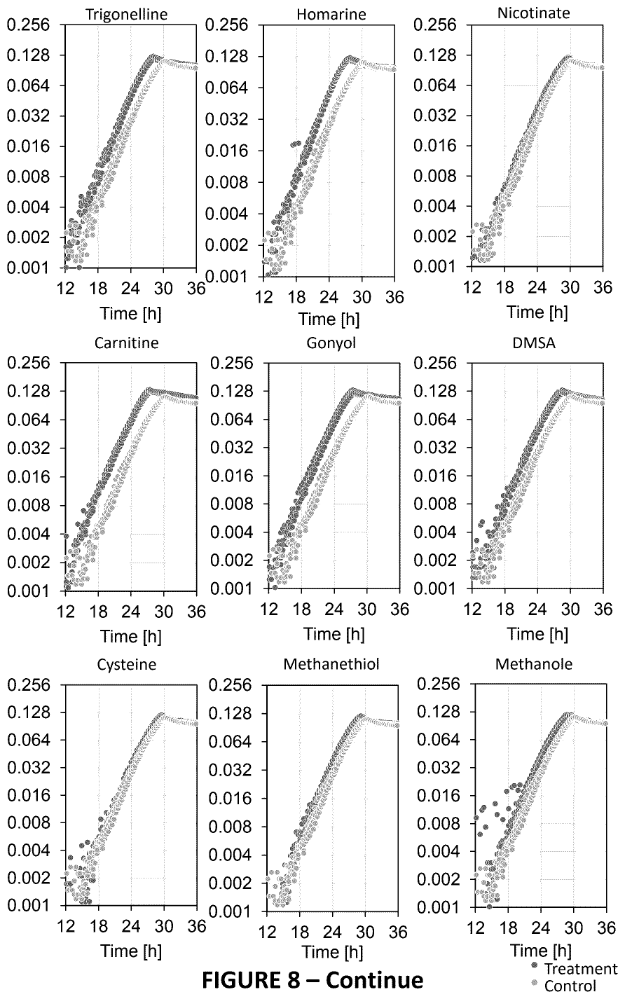

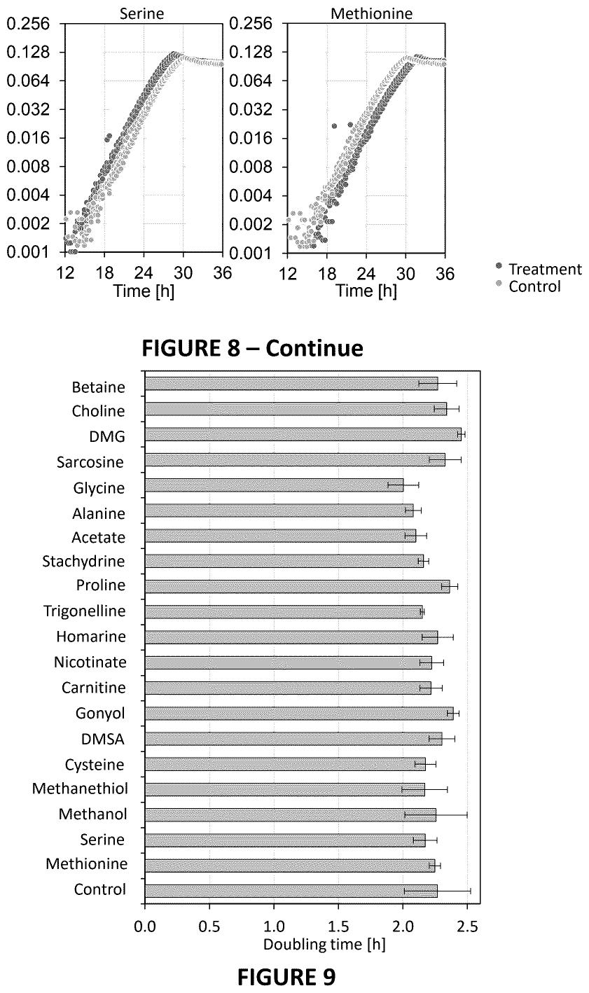

To test whether other methylated compounds shorten the bacterial lag phase, the inventors screened a panel of methylated molecules that are commonly produced by microalgae (FIGS. 7B and 8). The inventors selected compounds with varying chemical properties to assess their possible impact on lag phase shortening. Specifically, the inventors tested compounds that possess either an N-methylated amino group or an S-methylated sulfhydryl group. Additionally, small one-carbon compounds and amino acids were also tested. Of note are the amino acids serine and glycine, which do not carry a methyl group, yet can donate a one-carbon group to the cellular methyl group metabolism (FIG. 1C). All tested methylated metabolites were found to shorten the lag phase (FIG. 7B) while doubling times were roughly maintained (FIG. 9). The lag phase shortening effect was most pronounced for quaternary ammonium compounds with three methyl groups (such as carnitine, choline, betaine, etc.) or two methyl groups (such as stachydrine), as well as for sulfonium compounds with two methyl groups (including gonyol, and DMSA). The extent of the lag phase shortening correlated with the number of methyl groups per molecule, which is in line with the current results for DMSP and its analogs (FIG. 7A). Interestingly, the only exception was observed for methionine, which prolonged the lag phase. Growth inhibitions by methionine, which were previously reported in other bacteria, indicate the involvement of a negative feedback loop that regulates intracellular methionine concentrations. In summary, the current results revealed that a wide array of methylated compounds shorten the bacterial lag phase.

### Example 4

### Methyl Groups are a Limiting Resource During the Lag Phase

The inventors further sought to better understand why methyl groups shorten the bacterial lag phase. The inventors list at least three cellular scenarios which could explain the effect that methylated compounds have on the lag phase: (1) methyl groups might be a limiting resource; (2) methyl groups might act as a signal that triggers a cellular cascade, which culminates in lag phase shortening; and (3) methylated compounds could accumulate intracellularly and function as osmoprotectants and antioxidants. To test whether methyl groups are a limiting resource, the inventors generated P. inhibens knockout (KO) mutants that are no longer capable of harvesting methyl groups from specific donor compounds. Two KO mutants were generated: a strain in which the gene dmdA encoding a demethylase responsible for DMSP demethylation (FIG. 1C, gene 25) was deleted. In a second strain, the demethylation gene mttB1 (FIG. 1C, gene 3), which was previously associated with the demethylation of betaine under anaerobic conditions (Ticak et al., 2014), was deleted. Further, high expression of the mttB1 gene in the presence of microalgae indicates that it also plays a role under aerobic conditions (FIGS. 1B, gene 3). The inventors found that DMSP does not shorten the lag phase of the dmdA KO mutant. Similarly, betaine does not shorten the lag phase of the mttB1 KO mutant. The results highlight the centrality of demethylation genes in lag phase shortening. These observations indicate that methyl groups are actively harvested during the lag phase, and thus, suggest that methyl groups are a limiting resource.

To further assess whether methyl groups are utilized as a one-carbon building block, or to generate ATP, the inventors sought to estimate the bacterial methyl group requirements during the lag phase. The current experimental data show that as minimally as 2 nM of DMSP per 25,000 cells/mL (=OD 0.00001) triggered a significant lag phase shortening in bacteria cultivated with glucose as a main carbon source (FIG. 5C). This corresponds to 80 attomole (amol) of DMSP, or 160 amol of methyl groups per cell. Using theoretical numbers generated for E. coli (Neidhardt et al., 1990), the inventors estimated that a single P. inhibens cell requires 307 amol of one-carbon groups to synthesize key cellular components (i.e., purines, thymine, methionine and histidine; See Table 1).

TABLE 1
Theoretical methyl group requirements in P. inhibens

| | Building block¹ | | | |
|---|---|---|---|---|
| | ATP | GTP | dATP | dGTP |
| | (RNA) | (RNA) | (DNA) | (DNA) |
| Class | Purine | Purine | Purine | Purine |

Experimentally determined amount of building block in E. coli B/r³

| | | | | |
|---|---|---|---|---|
| [μmol/g | 165 | 203 | 24.7 | 25.4 |
| dried cells] | | | | |
| [amol/cell] | 46.2 | 56.8 | 6.9 | 7.1 |

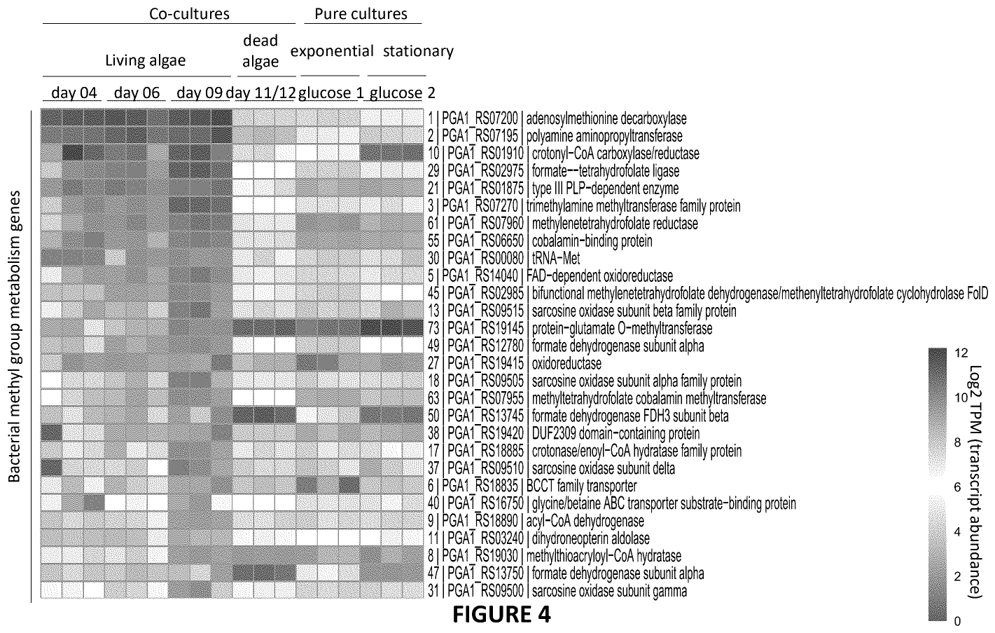

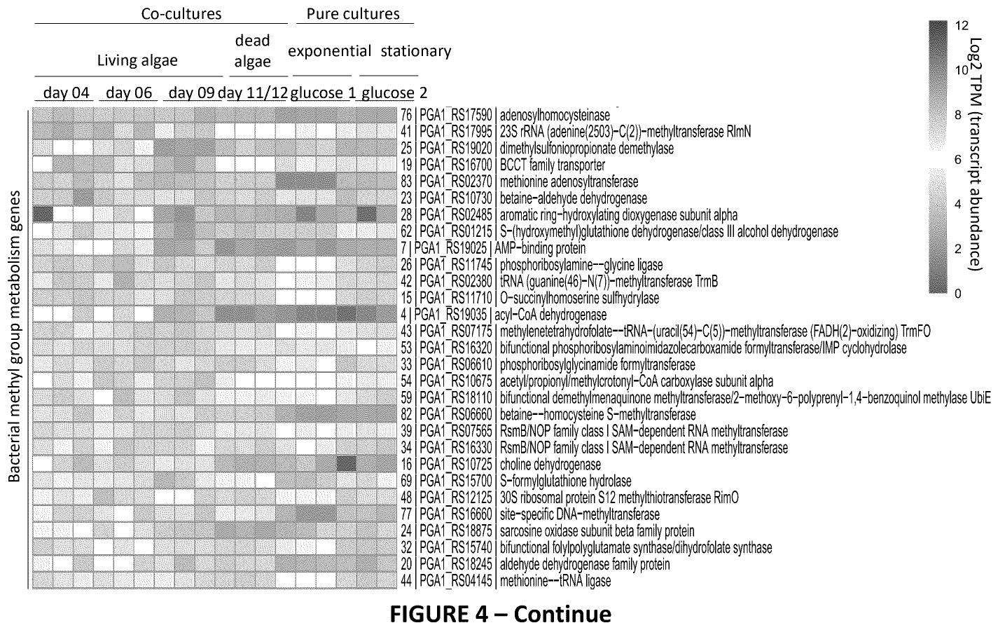

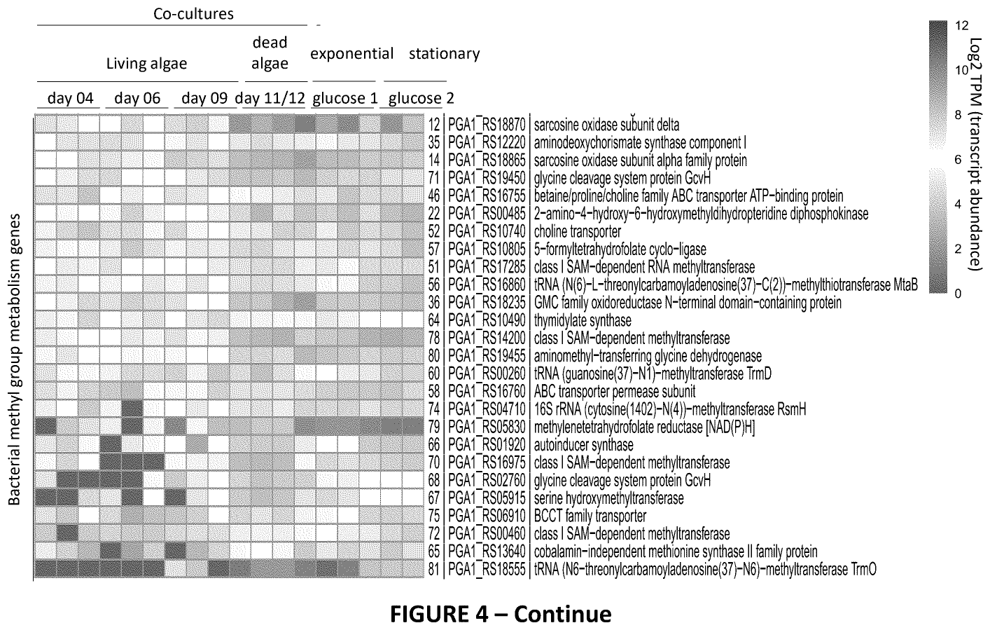

Theoretical methyl group requirement per building block in P. inhibens

| One-carbon groups | 2 | 2 | 2 | 2 |
|---|---|---|---|---|
| incorporated per | | | | |
| building block | | | | |
| Key enzyme(s) | 1: Phosphoribosyl- | 1: Phosphoribosyl- | 1: Phosphoribosyl- | 1: Phosphoribosyl- |
| | glycinamide | glycinamide | glycinamide | glycinamide |
| | formyltransferase | formyltransferase | formyltransferase | formyltransferase |
| | 2: Phosphoribosyl- | 2: Phosphoribosyl- | 2: Phosphoribosyl- | 2: Phosphoribosyl- |
| | aminoimidazole- | aminoimidazole- | aminoimidazole- | aminoimidazole- |
| | carboxamide | carboxamide | carboxamide | carboxamide |
| | formyltransferase | formyltransferase | formyltransferase | formyltransferase |
| One-carbon | 1: CHO-THF | 1: CHO-THF | 1: CHO-THF | 1: CHO-THF |
| group donor | 2: CHO-THF | 2: CHO-THF | 2: CHO-THF | 2: CHO-THF |
| Accession IDs | 1: purN, | 1: purN, | 1: purN, | 1: purN, |
| (gene name, | PGA1_RS06610, | PGA1_RS06610, | PGA1_RS06610, | PGA1_RS06610, |
| RefSeq locus | PGA1_c13270, | PGA1_c13270, | PGA1_c13270, | PGA1_c13270, |
| tag, submitter | WP_014874448.1 | WP_014874448.1 | WP_014874448.1 | WP_014874448.1 |
| locus tag, | 2: purH, | 2: purH, | 2: purH, | 2: purH, |
| protein) | PGA1_RS16320, | PGA1_RS16320, | PGA1_RS16320, | PGA1_RS16320, |
| | PGA1_c32860, | PGA1_c32860, | PGA1_c32860, | PGA1_c32860, |
| | WP_014881359.1 | WP_014881359.1 | WP_014881359.1 | WP_014881359.1 |
| Genes in | 1:33 | 1:33 | 1:33 | 1:33 |
| FIGS. 1, 4 | 2:53 | 2:53 | 2:53 | 2:53 |
| and 10-11 | | | | |
| One-carbon | 92.4 | 113.7 | 13.8 | 14.2 |
| requirement | | | | |
| [amol/cell]⁵ | | | | |
| | | Building block¹ | | |
| | | dTTP | Histidine² | Methionine |
| | | (DNA) | (protein) | (protein) |
| | Class | Thymine | Amino acid | Amino acid |
| | Experimentally determined amount of building block in E. coli B/r³ | | | |
| | [μmol/g | 24.7 | 90 | 146 |
| | dried cells] | | | |
| | [amol/cell] | 6.9 | 25.2 | 40.9 |
| | Theoretical methyl group requirement per building block in P. inhibens | | | |
| | One-carbon groups | 1 | 1 | 1 |
| | incorporated per | | | |
| | building block | | | |
| | Key enzyme(s) | Thymidylate | Phosphoribosyl- | Methionine |
| | | synthase | aminoimidazole- | synthase |
| | | | carboxamide | |
| | | | formyltransferase | |
| | One-carbon | CH₂-THF | CHO-THF | CH₃-cobalamin, |
| | group donor | | | betaine, DMSP⁴ |
| | Accession IDs | thyA, | purH, | bmt, |
| | (gene name, | PGA1_RS10490, | PGA1_RS16320, | PGA1_RS06660, |
| | RefSeq locus | PGA1_c21180, | PGA1_c32860, | PGA1_c13370, |
| | tag, submitter | WP_014880433.1 | WP_014881359.1 | WP_014879827.1 |
| | locus tag, | | | |
| | protein) | | | |
| | Genes in | — | 53 | 82 |
| | FIGS. 1, 4 | | | |
| | and 10-11 | | | |
| | One-carbon | 6.9 | 25.2 | 40.9 |
| | requirement | | | |
| | [amol/cell]⁵ | | | |

¹The listed building blocks are the major sinks for one-carbon groups in bacteria (Stauffer 2004). Not included is phosphatidylcholine, which is a lipid that is produced in many bacteria (Geiger et al., 2013), and that has a choline backbone with three N-methyl groups. It was reported that some Phaeobacter species produce phosphatidylcholine (Martens et al., 2006), however, only minor amounts of this lipid were detected in P. inhibens DSM 17395 when grown with glucose (Trautwein et al., 2018). Also not included are the methylated forms of the biomolecules DNA (Oliveira and Fang, 2021), RNA (Höfer and Jäschke, 2018) or proteins (Murn and Shi, 2017). These methylation modifications have regulatory or protective functions, and may constitute a major sink for one-carbon groups in the cell. However, to the best of the current inventors' knowledge, no robust data exist on the quantity of biomolecule methylations in the cell. Lastly, also the building block spermidine was not included in the table, which is a possible sink for one-carbon groups in P. inhibens. The bacterium possess the genes to synthesize spermidine by first decarboxylating S-adenosylmethionine (SAM) to S-adenosyl 3-(methylsulfanyl)propylamine (dcSAM), and then condensing dcSAM with either putrescine or agamtine. The decarboxylation of SAM to dcSAM is catalyzed by the speD gene product, which plays an important role in P. inhibens, as it is among the highest expressed metabolic genes during co-cultivation with algae and in the lag phase (FIGS. 1, 4, 10, and 12; gene 1). Of note, the S-methyl group of dcSAM is not directly incorporated into spermidine, but remains attached to a side product of spermidine synthesis, which is S-methyl-5′-thioadenosine (MTA; FIG. 12). The S-methyl group of MTA is recycled in many bacteria by the methionine salvage pathway; however, this pathway is incomplete in P. inhibens. The incomplete salvage pathway may result in the loss of one methyl group per synthesized spermidine molecule. However, since alternative methionine salvage pathways may exist that could recover the S-methyl group (Bullock et al., 2014), spermidine is not listed as a sink for one-carbon groups in the table.
²Histidine is synthesized by condensing the adenine backbone of ATP with the ribose sugar 5-phospho-α-D-ribose 1-diphosphate (PRPP). The side product of histidine synthesis is 5-amino-1-(5-phospho-D-ribosyl)imidazole-4-carboxamide (AICAR). AICAR is channeled into the lower branch of purine synthesis to regenerate the ATP, which consumes one formyl group per synthesized histidine molecule.
³Values were taken from Neidhardt et. al (1990). The numbers were given for E. coli cells grown at 37° C. in aerobic glucose minimal medium, at a doubling time of 40 min. The authors reported that a single E. coli cell has a dry weight of 280 pg under this condition. The inventors used this weight to calculate the amount of building block present per E. coli cell.
⁴Methionine synthesis is carried out in P. inhibens by the bmt gene product (Bmt), which is part of a split methionine synthase (Price et al., 2018). The Bmt enzyme was described to use CH3-cobalamine as methyl group donor (Price et al., 2018), however, it cannot be ruled out that the enzyme also uses other methyl group donors such as betaine or DMSP (Barra et al., 2006). Besides Bmt, P. inhibens encodes for a second methionine synthase, which is encoded by the metE gene (MetE), and that uses CH3-THF as methyl group donor. However, metE gene expression levels were low under all tested conditions (FIGS. 1A, 1C, and 4; gene 65), suggesting that MetE plays only a minor role.
⁵To calculate the one-carbon requirement per P. inhibens cell, the inventors multiplied the amount of building block produced per E. coli cell by the number of one-carbon groups that are required to synthesize the respective building block. This resulted in an estimated one-carbon requirement of 307.1 amol per cell. For this calculation, the inventors assumed that P. inhibens and E. coli cells produce the same amounts of the respective building blocks. This assumption is corroborated by the circumstance that P. inhibens is, like E. coli, a copiotrophic bacterium (Wünsch et al., 2019) with a rod-shaped form and a length of 1-2 μm (Martens et al., 2006).

These one-carbon groups can be generated from glucose either via the serine hydroxymethyltransferase (FIG. 1C; gene 67), or from the supplemented DMSP. Assuming that each P. inhibens cell harvests 160 amol methyl groups from DMSP during the lag phase, then this could cover 52% of the cellular one-carbon demand. Besides using the methyl groups of DMSP as building blocks, they could be also dissimilated for ATP generation (FIG. 1C; gene 29). E. coli has an estimated growth and maintenance cost of 19,040 amol ATP per cell during exponential growth with glucose (Feist et al., 2007; calculations were performed based on an estimated E. coli cell dry weight of 280 pg). Assuming similar ATP costs for P. inhibens, and that each cell transforms 160 amol of DMSP-derived methyl groups into 160 amol ATP during the lag phase, then the methyl groups could cover only 0.8% of the cellular ATP demand. Based on these calculations, it appears more plausible that methyl groups are utilized during the lag phase as a limiting building block, rather than as an ATP source.

### Example 5

### Methyl Groups are Assimilated During the Lag Phase

To further elucidate the fate of methyl groups, the inventors analyzed the transcriptome of DMSP-treated P. inhibens bacteria during the lag phase, and compared it to untreated control cultures. The results showed that the methyl group metabolism pathway was upregulated in DMSP-treated cells within the first 15 min (FIGS. 10A and 11A) and 40 min (FIG. 11B) of the lag phase. Specifically, the highest upregulated genes were involved in converting SAM into polyamines (FIG. 11A; genes 1, 2 and 21); a process that involves the methionine cycle (FIG. 10A). To further establish that DMSP methyl groups are assimilated via the methionine cycle, the inventors measured the methionine synthesis activity in protein crude extracts of stationary bacterial cells, using a biochemical assay followed by LC/MS analysis. This showed that methionine synthesis activity is elevated in bacteria treated with DMSP (FIG. 11B). By adding 13C-labeled DMSP to lag phase bacteria, it was further found that the DMSP methyl groups are converted into SAM, which is the precursor for polyamine synthesizes (FIG. 12). Taken together, the inventors established that DMSP methyl groups are assimilated during the lag phase via the methionine cycle.

### Example 6

### Bmt Catalyzes Methionine Biosynthesis Using DMSP as a Methyl Donor

To further establish the transfer of methyl groups from DMSP to the methionine cycle during the lag phase, the inventors proceeded to characterize the underlying enzymatic machinery. Previous studies identified specialized enzymes that can transfer methyl groups directly from a methylated compound onto homocysteine (Hcy), thus forming methionine in one step. Such enzymes were characterized in E. coli, in which the enzyme MmuM uses S-methyl-L-methionine (SMM; Thanbichler et al., 1999) as methyl group donor, and in humans, in which the enzyme BHMT1 uses betaine as methyl group donor (Li et al., 2016). Using amino acid sequence similarity, the inventors identified a homologues enzyme in P. inhibens, which is a betaine-homocysteine S-methyltransferase (Bmt) that is encoded by the bmt gene. To test whether the bmt-encoded enzyme can directly transfer methyl groups from DMSP onto Hcy, the inventors conducted an in vitro methionine synthesis assay. For this assay, Bmt was expressed in a heterologous system and purified (FIG. 13). The purified Bmt was then incubated with DMSP and hcy, and a fluorescent assay was used to measure methionine formation. The current data reveal that Bmt indeed catalyzes the production of methionine by transferring a methyl group directly from DMSP onto Hcy (FIG. 14).

The result demonstrates that the P. inhibens Bmt can transfer a methyl group directly from DMSP onto Hcy to generate methionine.

### Example 7

### Bmt is Required for Lag Phase Shortening

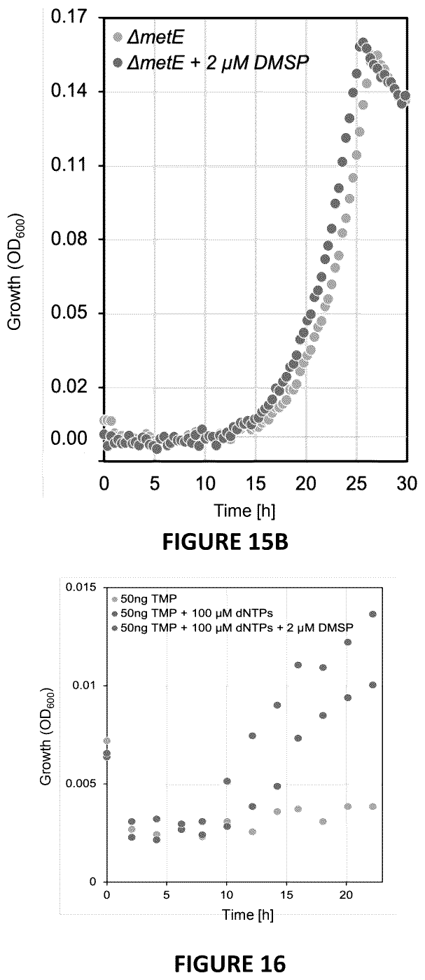

To corroborate the involvement of Bmt in lag phase shortening, the inventors deleted the bmt gene in P. inhibens. The Δbmt mutant showed impaired growth in minimal media without methionine, with minor growth only after 30 hours. The delayed minor growth may indicate the activation of redundant and less efficient methionine synthases, a common trait among bacteria (Husna et al., 2018). The growth of the Δbmt was fully recovered upon addition of 200 μM methionine. In both conditions, either with or without methionine, the addition of 2 μM DMSP did not induce lag phase shortening (FIG. 15A). Thus, Bmt appears to be a central methionine synthase that is involved in lag phase shortening.

P. inhibens harbors another methionine synthase encoded by metE (FIGS. 1C and 4; gene 65). To test the possible contribution of this enzyme to lag phase shortening the inventors knocked-out the gene and examined the performance of the mutant strain. The ΔmetE mutant exhibited lag phase shortening similar to wild-type bacteria upon exposure to 2 μM DMSP (FIGS. 15B and 5B, respectively). To further rule out the involvement of MetE in lag phase shortening, the inventors perturbed the function of MetE using an inhibitor of its methyl donor. To produce methionine, MetE utilizes 5-methyl-tetrahydrofolate (THF) as a methyl donor (Pejchal and Ludwig, 2005). THF is central in the process of bacterial nucleotide production (Quinlivan et al., 2000). In the presence of the THF inhibitor Trimethoprim (TMP), P. inhibens bacteria cannot grow unless the culture is supplemented with nucleotides. Under these conditions, addition of 2 μM DMSP still elicited lag phase shortening (FIG. 16). These data show that MetE is not involved in lag phase shortening and that Bmt is the key methionine synthase in this process.

### Example 8

### Methylated Compounds Shorten the Lag Phase in Various Bacteria

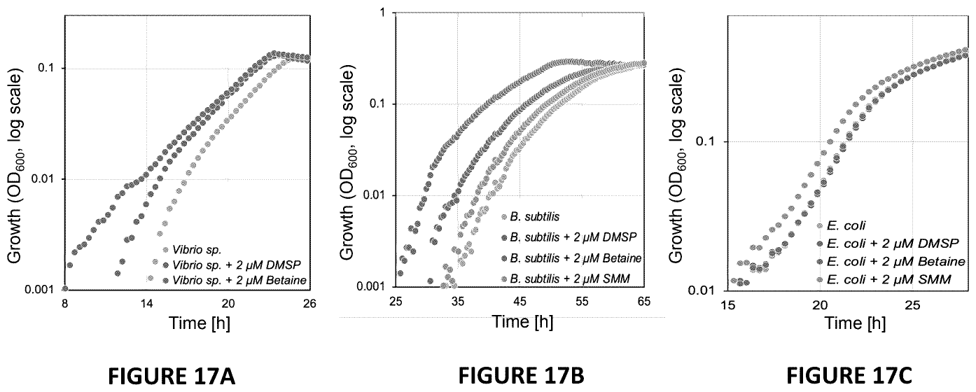

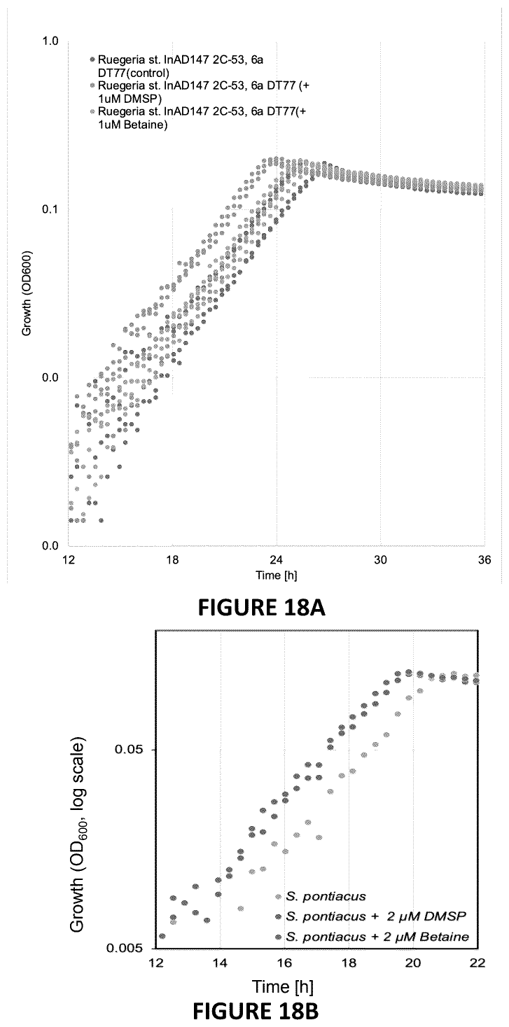

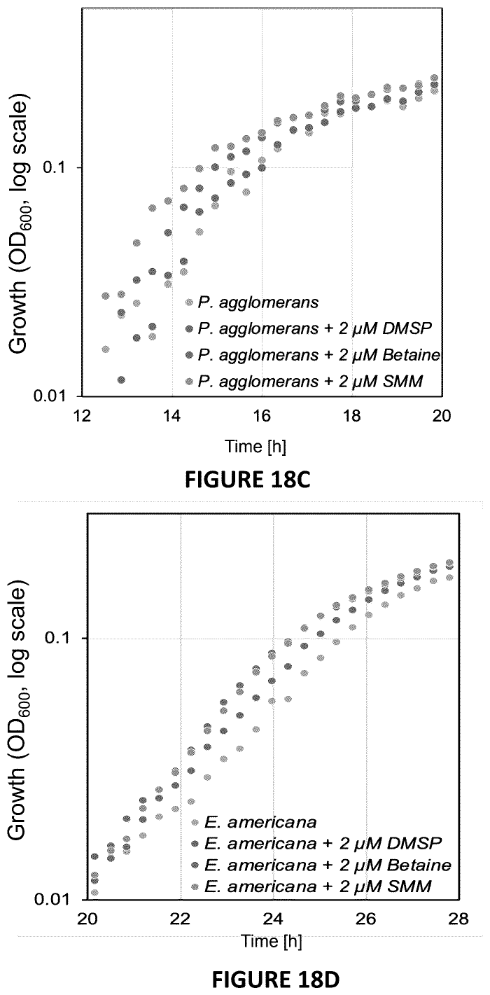

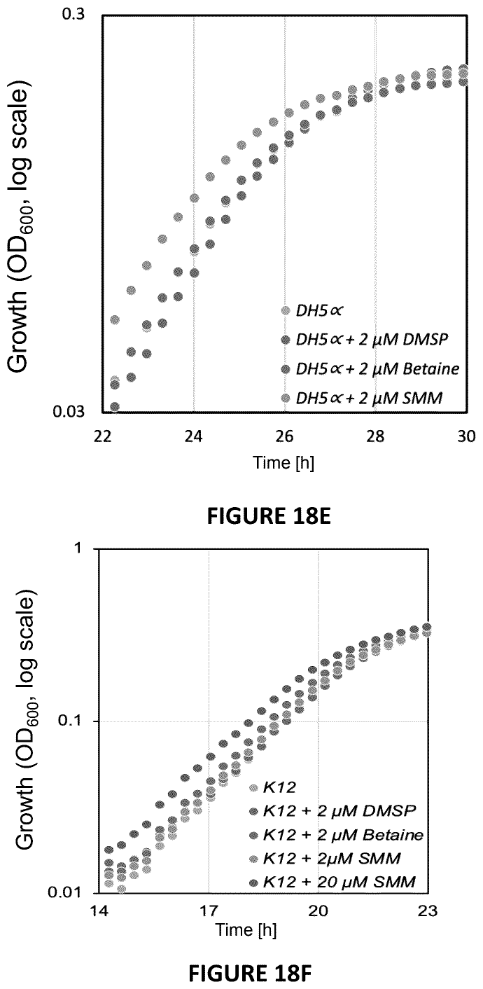

To explore whether lag phase shortening is a general mechanism among bacteria, the inventors tested the effect of methylated compounds in bacteria with different lifestyles. Various marine bacteria exhibited marked lag phase shortening upon exposure to minute amounts of DMSP or betaine (FIGS. 17A and 18). Since methylated compounds are a hallmark of photosynthesizing organisms, the inventors next tested a variety of bacteria that are plant-associated. The current results reveal significant shortening of the lag phase in these bacteria upon treatment with betaine, and to slightly less extent with DMSP. Of note, the model plant-associated bacterium B. subtilis exhibited a Δlag time of 7 hours when treated with betaine (FIG. 17B). Some opportunistic pathogens, found in both plants and humans, also shorten their lag phase in response to S-methyl-L-methionine (SMM), the main available methyl donor in plants (Bourgis et al., 1999). Finally, to establish the prevalence of lag phase shortening among bacteria, the inventors tested the model bacterium E. coli. This bacterium is not commonly found associated with photosynthesizing organisms, and thus, is unlikely to respond to algal metabolites. Indeed, treatment with either DMSP or betaine did not shorten the lag phase of E. coli. However, treatment of E. coli with the bona fide methyl donor SMM, resulted in a Δlag time of one hour (FIG. 17C). Interestingly this effect was observed in E. coli modified strains BL21(DE3), and DH5α, while wild-type strain K12 only showed this effect upon addition of greater SMM concentrations (FIGS. 17C and 18). The current data suggest that lag phase shortening by methylated compounds is a common trait among bacteria. The synchrony between specific compounds and different groups of bacteria may reflect the natural context under.

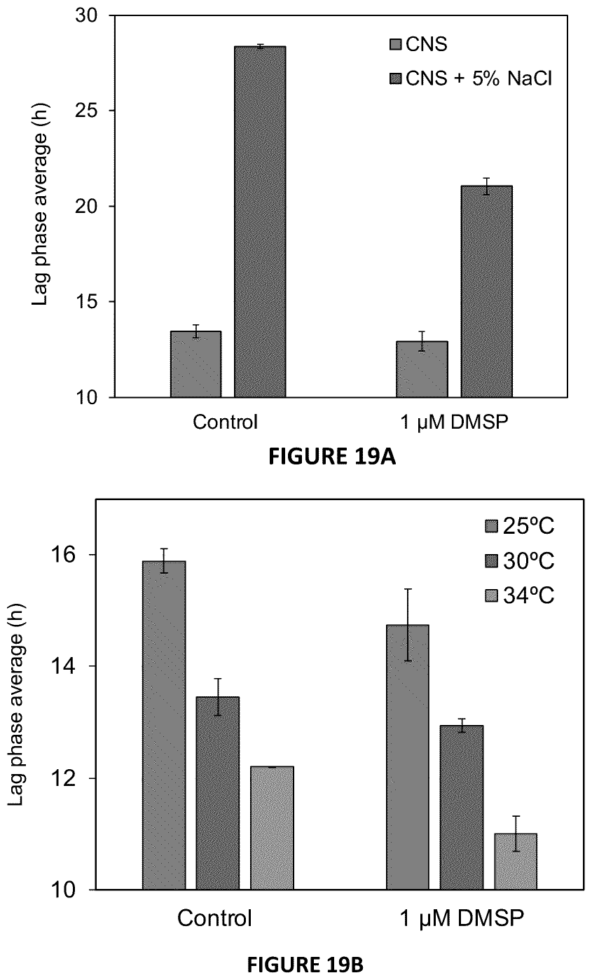

Also, the inventors have shown that the lag phase shortening effect is further pronounced under high salt conditions (FIG. 19A), and in temperatures that deviate from the ‘30° C. optimum’ of Phaeobacter inhibens (FIG. 19B).

Therefore, the inventors suggest that shortening lag phase of a microorganism can be achieved, by contacting the microorganism with an effective amount of a methyl group donor, such as including a tertiary sulfonium group or a quaternary ammonium group, and/or one-carbon group donor.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims which follow.

1. A method for shortening lag phase of a microorganism, the method comprising contacting said microorganism with an effective amount of a methyl group donor, thereby shortening lag phase of the microorganism.

2. The method of claim 1, wherein said methyl group donor comprises a tertiary sulfonium group or a quaternary ammonium group.

3. The method of claim 1, wherein said methyl group donor comprises one or more methyl groups.

4. The method of claim 3, wherein said one or more methyl groups is covalently bound to a sulfur atom or a nitrogen atom.

5. The method of claim 1, wherein said methyl group donor is selected from the group consisting of: dimethylsulfoniopropionate (DMSP), betaine, choline, dimethylsulfonioacetate (DMSA), carnitine, homarine, stachydrine, trigonelline, gonyol, S-methylmethionine (SMM), and any combination thereof.

6. The method of claim 2, wherein said methyl group donor comprising a tertiary sulfonium group is selected from the group consisting of: DMSP, DMSA, gonyol, and any combination thereof.

7. The method of claim 2, wherein said methyl group donor comprising a quaternary ammonium group is selected from the group consisting of: betaine, choline, homarine, carnitine, stachydrine, trigonelline, and any combination thereof.

8. The method of claim 1, further comprising a step comprising subjecting said microorganism to suboptimal salt concentration, suboptimal temperature, or both.

9. The method of claim 1, wherein said microorganism is a transgenic cell or a transformed cell.

10. The method of claim 9, wherein said transgenic cell or transformed cell heterologously expresses a polynucleotide encoding a betaine-homocysteine S-methyltransferase (Bmt).

11. The method of claim 1, wherein said microorganism is selected from the group consisting of: bacterium, fungus, microalga, and any combination thereof.

12. The method of claim 11, wherein said fungus is a yeast.

13. The method of claim 1, further comprising a step preceding said contacting, comprising transfecting or transforming said microorganism with a polynucleotide encoding a Bmt.

14. The method of claim 10, wherein said polynucleotide comprises a nucleic acid sequence set forth in SEQ ID NO: 1, or a functional analog thereof having at least 80% sequence homology thereto.
